(19) United States
(12) Patent Application Publication
(10) Pub. No.: US 2022/0228101 A1
(43) Pub. Date: Jul. 21, 2022

(54) AUTOMATED T CELL CULTURE

(71) Applicant: JUNO THERAPEUTICS, INC., Seattle, WA (US)

(72) Inventors: Ivie AIFUWA, Seattle, WA (US); Pascal BEAUCHESNE, Seattle, WA (US); Kien KHUU-DUONG, Snoqualmie, WA (US); Kohana LEUBA, Seattle, WA (US)

(73) Assignee: JUNO THERAPEUTICS, INC., Seattle, WA (US)

(21) Appl. No.: 17/617,257

(22) PCT Filed: Jun. 5, 2020
(86) PCT No.: PCT/US2020/036442

§ 371 Date: Dec. 7, 2021

Related U.S. Application Data
(60) Provisional application No. 62/858,736, filed on Jun. 7, 2019.

(51) Int. Cl. C12M 1/36; C12N 5/0783; C12N 15/86; C12N 15/62; C12M 1/26; C12M 1/34; G01N 35/00; A01N 1/02

(52) U.S. Cl. CPC C12M 41/48; C12N 5/0636; C12N 15/86; C12N 15/625; C12M 33/00; C12M 41/46; G01N 2035/00564; G01N 35/0099; A01N 1/0252; C12N 2740/10043; C12N 2740/15043; G01N 2035/00495; C12M 41/44

(57) ABSTRACT

An automated method of T cell scale down processing. The method including: activating T cells by automatically contacting the T cells with one or more activation reagents; transducing the T cells by automatically contacting the T cells with a recombinant viral vector; automatically inoculating T cells; automatically expanding the T cells; optionally, automatically debeading the T cells; and automatically harvesting the T cells. A system for an automated method of T cell scale down processing.

### CROSS-REFERENCE TO RELATED APPLICATION

This application claims the priority benefit of the earlier filing date of U.S. Provisional Patent Application No. 62/858,736, filed Jun. 7, 2019, which is hereby incorporated herein by reference in its entirety.

### REFERENCE TO A SEQUENCE LISTING

This application incorporates by reference the Sequence Listing submitted in Computer Readable Form created on May 27, 2020 and containing 18 kilobytes.

### FIELD

This disclosure relates generally to the field of cell culture. In particular, this disclosure relates to systems and methods for small scale automated culture, and genetic modification of mammalian cells, such as T cells.

### BACKGROUND

Various cell therapy methods are available for treating diseases and conditions. Among cell therapy methods are methods involving immune cells, such as T cells, genetically engineered with a recombinant receptor, such as a chimeric antigen receptors. Improved methods for manufacturing and/or engineering such cell therapies are needed, including to provide for a more efficient method of testing various conditions and genetically engineered T cells.

### SUMMARY

One aspect of the present disclosure is as an automated method of T cell scale-down manufacturing, in which the method includes: activating, an input set of T cells by contacting the input set of T cells obtained from one or more donors with one or more activation reagents to generate a set of activated T cells; transducing the set of activated T cells by contacting the activated T cells with a recombinant viral vector under conditions that promote viral infection of the activated T cells, wherein the recombinant viral vector comprises a nucleic acid that encodes a heterologous recombinant protein; inoculating and/or incubating the set of transduced T cells by transferring the set of activated T cells into inoculation and/or incubation media expanding the set of transduced T cells by recovering the set of transduced T cells from the inoculation and/or incubation media and transferring the set of transduced T cells into expansion media; recovering the set of transduced T cells from the expansion media; and harvesting the set transduced T cells by cryopreserving the set of transduced T cells to generate a harvested set of transduced T cells.

In some embodiments, the method further includes setting up a worktable with labware and one or more activation reagents.

In any of the above embodiments, one or more steps may be performed automatically. For example, automatically contacting the input set of T cells obtained from one or more donors with one or more activation reagents, automatically contacting the activated T cells with a recombinant viral vector, automatically transferring the set of activated T cells into inoculation and/or incubation media, automatically recovering the set of transduced T cells from the inoculation media and transferring the set of transduced T cells into expansion media, automatically recovering the set of transduced T cells from the expansion media and/or automatically cryopreserving the set transduced T cells. In some embodiments, the present disclosure relates to an automated method of T cell scale-down manufacturing, in which the method includes: activating, an input set of T cells by automatically contacting the input set of T cells obtained from one or more donors with one or more activation reagents to generate a set of activated T cells; transducing the set of activated T cells by automatically contacting the activated T cells with a recombinant viral vector under conditions that promote viral infection of the activated T cells, wherein the recombinant viral vector comprises a nucleic acid that encodes a heterologous recombinant protein; inoculating and/or incubating the set of transduced T cells by automatically transferring the set of activated T cells into inoculation and/or incubation media expanding the set of transduced T cells by automatically recovering the set of transduced T cells from the inoculation media and transferring the set of transduced T cells into expansion media; automatically recovering the set of transduced T cells from the expansion media; and harvesting the set transduced T cells by automatically cryopreserving the set transduced T cells to generate a harvested set of transduced T cells.

In various embodiments of the method, that may be combined with any other embodiment, transducing comprises: obtaining samples of the set of activated T cells for viable cell counting; preparing the set of activated T cells for spinoculation; spinoculating the set of activated T cells by contacting the set of activated T cells with the recombinant viral vector and applying a centrifugal force to the set of activated T cells; and, incubating and/or inoculating the set of activated T cells in an mammalian cell incubator post transduction. In some embodiments, any one or more of the above steps may be performed automatically. For example: automatically obtaining samples of the set of activated T cells for viable cell counting; automatically preparing the set of activated T cells for spinoculation; automatically spinoculating the set of activated T cells by contacting the set of activated T cells with the recombinant viral vector and applying a centrifugal force to the set of activated T cells; and, automatically incubating and/or inoculating the set of activated T cells in an mammalian cell incubator post transduction.

In some embodiments, the method further includes setting up the worktable with the labware and reagents for the transduction of the set of activated T cells.

In various embodiments of the method, inoculating comprises: obtaining samples of the set of activated T cells after transducing for viable cell counting; and inoculating the set of activated T cells by automatically transferring the set of activated T cells to expansion plates and placing the expansion plates containing the set of activated T cells in mammalian cell incubator. In some embodiments, any one or more of the above steps may be performed automatically. For example, automatically obtaining samples of the set of activated T cells after transducing for viable cell counting.

In some embodiments, the method further includes setting up a worktable with the labware and reagents for inoculating the set of activated T cells.

In various embodiments of the method, expanding further comprises: obtaining samples of the set of transduced T cells for viable cell counting; and performing mock perfusion/cell culture media exchange. In some embodiments, any one or more of the above steps may be performed automatically. For example: automatically performing mock perfusion/cell culture media exchange.

In some embodiments, the method further includes setting up a worktable with the labware and reagents for expanding the set of transduced T cells.

In various embodiments of the method, debeading comprises: obtaining samples prior to the debeading step for viable cell counting; debeading the set of transduced T cells by applying a magnetic field; and optionally, obtaining samples after the debeading step for viable cell counting. In some embodiments, any one or more of the above steps may be performed automatically. For example: automatically obtaining samples prior to the debeading step for viable cell counting; automatically debeading the set of transduced T cells by applying a magnetic field; and optionally, automatically obtaining samples after the debeading step for viable cell counting.

In some embodiments, the method further includes setting up a worktable with the labware and reagents for debeading.

In various embodiments of the method, harvesting comprises: placing the set of transduced T cells in cryovials with cryopreservation media; and placing the cryovials in a liquid nitrogen tank.

In some embodiments, the method further includes setting up a worktable with the labware and reagents for cryopreserving transduced T cells.

In various embodiments of the method, the T cells comprise CD4+ T cells.

In various embodiments of the method, the T cells comprise CD8+ T cells.

In various embodiments of the method, the T cells comprise CD4+ T cells and CD8+ T cells.

In various embodiments of the method, the heterologous recombinant protein comprises a recombinant receptor.

In various embodiments of the method, the recombinant receptor is capable of binding to a target antigen that is associated with, specific to, and/or expressed on a cell or tissue of a disease, disorder or condition.

In various embodiments of the method, the disease, disorder or condition is an infectious disease or disorder, an autoimmune disease, an inflammatory disease, or a tumor or a cancer.

In various embodiments of the method, the target antigen is a tumor antigen.

In various embodiments of the method, the recombinant receptor is or comprises a functional non-T cell receptor (TCR) antigen receptor or a TCR or antigen-binding fragment thereof.

In various embodiments of the method, the recombinant receptor is a chimeric antigen receptor (CAR).

In various embodiments of the method, the viral vector comprises is a retroviral vector.

In various embodiments of the method, the viral vector is a lentiviral vector or gammaretroviral vector.

In various embodiments of the method, the T cells comprises primary T cells obtained from one or more donors.

In various embodiments of the method, the one or more donors is a human subject.

With regard to the disclosed methods, reference is made to transducing the set of activated T cells by contacting the activated T cells with a recombinant viral vector under conditions that promote viral infection of the activated T cells. However, it is contemplated that the disclosed methodology can include non-viral methods of incorporation of nucleic acids that encode the heterologous recombinant protein. Examples of non-viral methodology for nucleic acid incorporation into the set of activated T cells may include, but are not limited to, electroporation, reagent-based transfection, cell compression, or squeezing. It is contemplated that non-viral incorporation of the nucleic acid can be performed automatically, for example, by automatic electroporation, automatic reagent-based transfection, automatic cell compression, automatic squeezing, etc., without departing from the scope of this disclosure.

Accordingly, in various embodiments, an automated method for T cell scale down processing as herein disclosed, includes activating an input set of T cells by automatically contacting the input set of T cells obtained from one or more donors (such as, one or more human donors) with one or more activation reagents to generate a set of activated T cells; modifying the set of activated T cells to generate a set of modified T cells by contacting the set of activated T cells with a recombinant polynucleotide under conditions that promote incorporation of the recombinant polynucleotide into the set of activated T cells, wherein the recombinant polynucleotide comprises a nucleic acid that encodes a heterologous recombinant protein; expanding the set of modified T cells in an expansion media; recovering the set of modified T cells from the expansion media; and harvesting the set of modified T cells by automatically cryopreserving the set of modified T cells to generate a harvested set of modified T cells.

In various embodiments of the method, modifying the set of activated T cells to generate the set of activated T cells further includes incorporating the recombinant polynucleotide via at least one of transduction, electroporation, reagent-based transfection, cell compression, or squeezing.

In various embodiments of the method, one or more of activating the input set of T cells, modifying the set of activated T cells, expanding the set of modified T cells, recovering the set of modified T cells, and harvesting the set of modified T cells is performed automatically, without intervention from an operator.

For example, in some embodiments of the method, the method further includes setting up a worktable with one or more of labware and/or reagents for the transduction, electroporation, reagent-based transfection, cell compression or squeezing to incorporate the recombinant polynucleotide into the set of activated T cells. The setting up of the worktable may be performed automatically, or at least partially automatically, in some examples.

In some examples of the method, the method further includes inoculating and/or incubating the set of modified T cells. For example, the set of modified T cells may be automatically transferred into inoculation and/or incubation media. The method may further include expanding the set of modified T cells by automatically transferring the set of modified T cells to expansion media. In some examples, the method may include setting up the worktable for the inoculation and/or expansion procedures. Setting up the worktable for the inoculation and/or expansion procedures may be performed automatically, or at least partially automatically, in some examples.

In some examples of the method, the method includes a debeading step. The debeading step may include obtaining samples prior to the debeading step for viable cell counting, and based on the cell counting, debeading the set of modified T cells by application of a magnetic field, in a case where the beads are attracted to, or responsive to, a magnetic field. In some examples, samples are obtained after the debeading for viable cell counting procedures. The sampling, and/or the debeading steps may be performed automatically, or at least partially automatically, in some examples. In some examples the worktable is set up for the steps of debeading and/or cell counting/sampling, and the worktable setup is done automatically or at least partially automatically, in examples.

In some examples of the method, the method includes setting up the worktable with labware and/or reagents for recovering and/or harvesting the set of modified T cells. The setting up of the worktable for recovering and/or harvesting the set of modified T cells may be done automatically, or at least partially automatically, in some examples. In some examples, harvesting the set of modified T cells includes placing the set of modified T cells in cryovials with cryopreservation media. In some examples, harvesting the set of modified T cells further includes placing the cryovials in a liquid nitrogen tank, in some examples.

In some examples of the method, the input set of T cells includes CD4+ T cells.

In some examples of the method, the input set of T cells includes CD8+ T cells.

In some examples, the T cells include CD4+ T cells and CD8+ T cells.

In some examples of the method, the heterologous recombinant protein includes a recombinant receptor. As one example, the recombinant receptor is capable of binding to a target antigen that is associated with, specific to, and/or expressed on a cell or tissue of an associated disease, disorder or condition. For example, the disease, disorder or condition may be one or more of an infectious disease or disorder an autoimmune disease, an inflammatory disease, a tumor or a cancer. In examples, the target antigen is a tumor antigen. In some examples, the recombinant receptor is a functional non-T cell antigen receptor, or an antigen-binding fragment of a T cell receptor. In some examples, the recombinant receptor is a chimeric antigen receptor (CAR).

Another aspect of the disclosure is a multiplex automated system for T cell scale down manufacturing. The system includes, in some embodiments, an automated liquid handling system, and a control system in communication with the automated liquid handling system, comprising one or more processers programmed to control the automated liquid handling system to perform the unit processes of: activating a set of T cells; modifying, such as by transducing, the set of T cells; debeading the set T cells; inoculating the set of T cells; expanding the set of T cells; and harvesting the set of T cells.

In various embodiments of the system, the automated liquid handling system comprises a flexible channel liquid manipulation module configured to transfer liquid in an independent multichannel pipette format, wherein each pipette is configured to be independently operated.

In various embodiments of the system, the flexible channel liquid manipulation module is configured to accurately manipulate fluid volumes between about 0.5-5000 μL based on a determination of liquid class.

In various embodiments of the system, the flexible channel liquid manipulation module is configured to use disposable tips to provide for a sterile culture.

In various embodiments of the system, the flexible channel liquid manipulation module is a liquid displacement flexible channel arm.

In various embodiments of the system, the automated liquid handling system is comprised of a static multichannel liquid manipulation module configured to transfer liquid in a multichannel pipette format.

In various embodiments of the system, the static multichannel liquid manipulation module is a multichannel arm.

In various embodiments of the system, the automated liquid handling system comprises a container manipulation module, with interchangeable gripper configurations.

In various embodiments of the system, the interchangeable gripper configurations comprise: eccentric fingers configured for horizontal access and transport of labware; centric fingers configured for vertical access to labware; and tube fingers configured for the transport of tube type labware.

In various embodiments of the system, the container manipulation module is a long z-axis robotic gripper arm.

In various embodiments of the system, the automated liquid handling system comprises a worktable independently configurable for activation, transduction, inoculation, expansion, debeading and harvest unit operations.

In various embodiments of the system, the automated liquid handling system comprises a temperature controlled robotic centrifuge.

In various embodiments of the system, the automated liquid handling system comprises a vial gripper module configured to hold and grip round labware.

In various embodiments of the system, the automated liquid handling system comprises an automated cell counting module, configured to take viable cell count measurements.

In various embodiments of the system, the automated liquid handling system comprises a portable cryovial cooling chamber/cap holder configured to hold cryovials.

In various embodiments of the system, the automated liquid handling system provides a sterile environment.

In some embodiments, the system further includes a mammalian cell incubator.

It is to be understood that the above-described system may be used to carry out any of the methods herein disclosed.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 is a schematic block diagram of an automated multiplex mammalian cell culture system, in accordance with embodiments disclosed herein.

FIG. 2 is a schematic of a liquid displacement Flexible Channel Arm (FCA).

FIG. 3 is a schematic of a Multiple Channel Arm (MCA).

FIG. 4A is a digital image of a top side of a 96 channel adapter.

FIG. 4B is a digital image of a bottom side of the 96 channel adapter shown in FIG. 4A.

FIG. 5 is a schematic of a Robotic Gripper Arm Long (RGA).

FIG. 6 is a schematic of eccentric fingers for the Robotic Gripper Arm Long (RGA) shown in FIG. 5.

FIG. 7 is a schematic of centric fingers for the Robotic Gripper Arm Long (RGA) shown in FIG. 5.

FIG. 8 is a schematic of tube fingers for the Robotic Gripper Arm Long (RGA) shown in FIG. 5.

FIG. 9 is digital image of a syringe configuration for the FCA shown in FIG. 2.

FIG. 10 is a schematic of a 7 mm microplate nest segment and 7 mm nest.

FIG. 11 is a schematic of a 100 mL reagent trough.

FIG. 12 is a schematic of a 50 mL conical tube runner.

FIG. 13 is a schematic of a 6 position hotels 105.

FIG. 14 is a schematic of a robotic centrifuge.

FIG. 15 is a schematic of a worktable 60 showing the configuration of the named components.

FIG. 16 is a schematic of a liquid handing system showing the configuration of the FCA, MCA, and RGA shown in FIGS. 2, 3, and 5, respectively.

FIG. 17 is a schematic of a workflow for an automated method of T cell culture, in accordance with certain embodiments.

### DETAILED DESCRIPTION OF DISCLOSED EMBODIMENTS

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, summary or the following detailed description.

This specification includes references to “one embodiment” or “an embodiment.” The appearances of the phrases “in one embodiment” or “in an embodiment” do not necessarily refer to the same embodiment. Particular features, structures, or characteristics may be combined in any suitable manner consistent with this disclosure.

Terminology. The following paragraphs provide exemplary descriptions of and/or context for terms found in this disclosure (including the appended claims):

“Comprising.” This term is open-ended. As used in the appended claims, this term does not foreclose additional structure or steps.

“Configured To.” Various units or components may be described or claimed as “configured to” perform a task or tasks. In such contexts, “configured to” is used to connote structure by indicating that the units/components include structure that performs those task or tasks during operation. As such, the unit/component can be said to be configured to perform the task even when the specified unit/component is not currently operational (e.g., is not on/active). Reciting that a unit/circuit/component is “configured to” perform one or more tasks is expressly intended not to invoke 35 U.S.C. § 112, sixth paragraph, for that unit/component.

“First,” “Second,” etc. As used herein, these terms are used as labels for nouns that they precede, and do not imply any type of ordering (e.g., spatial, temporal, logical, etc.).

“Coupled”—The following description refers to elements or nodes or features being “coupled” together. As used herein, unless expressly stated otherwise, “coupled” means that one element/node/feature is directly or indirectly joined to (or directly or indirectly communicates with) another element/node/feature, and not necessarily mechanically.

In addition, certain terminology may also be used in the following description for the purpose of reference only, and thus are not intended to be limiting. For example, terms such as “upper,” “lower,” “above,” “below,” “in front of,” and “behind” refer to directions in the drawings to which reference is made. Terms such as “front,” “back,” “rear,” “side,” “outboard,” “inboard,” “leftward,” and “rightward” describe the orientation and/or location of portions of a component, or describe the relative orientation and/or location between components, within a consistent but arbitrary frame of reference which is made clear by reference to the text and the associated drawings describing the component(s) under discussion. Such terminology may include the words specifically mentioned above, derivatives thereof, and words of similar import.

In the following description, numerous specific details are set forth, such as specific operations, in order to provide a thorough understanding of embodiments of the present disclosure. It will be apparent to one skilled in the art that embodiments of the present disclosure may be practiced without these specific details. In other instances, well-known techniques are not described in detail in order to not unnecessarily obscure embodiments of the present disclosure. The section headings used herein are for organizational purposes only and are not to be construed as limiting the subject matter described

Introduction

Producing genetically engineered T cells, such as CD4+ T cells and/or CD8+ T cells, for use in cell therapy is a multi-step process comprising a number of variables. For example, to produce genetically engineered T cells, the cells are subjected to incubation under stimulating conditions, introduction of a recombinant polypeptide to the cells through transduction, and cultivating the cells under conditions that promote proliferation and/or expansion. Each of these processes may be subject to variation, both in conditions tested and user/operator variably. Further, current T cell scale down tests may be limited by the number of resourced operators, and the maximum number of conditions the operator can perform at a given time. Tests may also be exposed to variability and inconsistencies due to operator handling and pipetting inaccuracies. These variabilities can lead to inconsistencies in results. To reduce the inconsistences associated with user/operator inputs and to aid in increasing developmental throughput, an automated scaled down T cell culture platform is needed. The automated scale down platform would provide a standardized T cell culture platform and improve consistency of scale down experimentation. This platform would be beneficial for routine testing such as raw material verification or with complex tasks such as media development. Additional methods can be written to allow for tasks like media and culture supplement screens and large design of experiments (DoE) experimental designs. To meet the above need, the inventors have developed an automated benchtop cell culture system and methods of using this system to facilitate the development of genetically engineered therapeutic T cells.

Example System

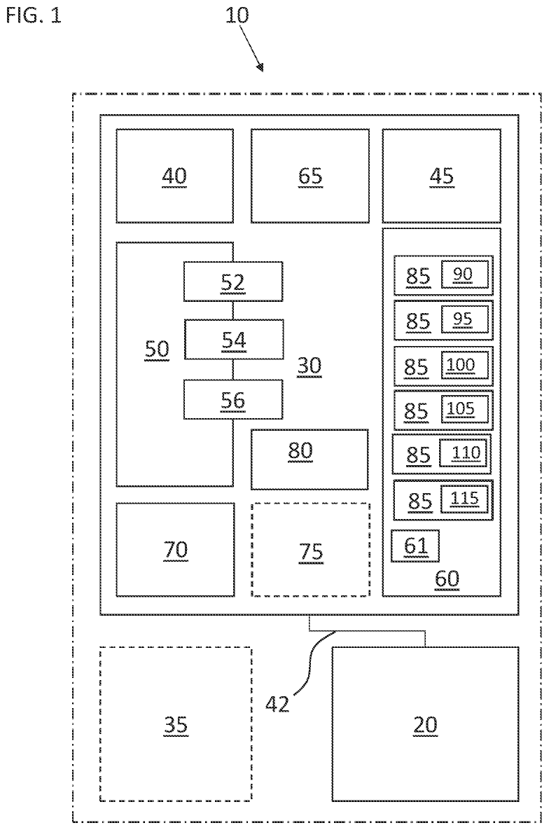

With reference to FIG. 1, disclosed herein is an automated cell culture system 10, which may be referred to as an automated T cell scale down model system. The system 10 includes a control system 20, such as a computer implemented control system, and an automated liquid handling system 30. As shown, the control system 20 and the liquid handling system 30 are connected by network 42. The system may also include an optional mammalian cell incubator 35. Network 42 can be any network, including a local area network (LAN) or a wide area network (WAN), or the connection may be made to an external computing device, (for example, through the Internet using an Internet Service Provider), or wireless network, or even as a direct connection, for example as an integrated component of the system 10. The control system 20 controls the various modules of the automated liquid handling system 30. In certain embodiments, the automated liquid handling system 30 includes a sterile environment, for example for sterile cell culture work, and may be contained in a housing having filters and/or positive ventilation to prevent contamination, for example a hood or cabinet. The liquid handling system 30 is composed of multiple modules for the manipulation of a liquid, liquids and containers comprising liquids, that may include mammalian cells of interest, such as T cells, for example CD4+ T cells and/or CD8+ T cells.

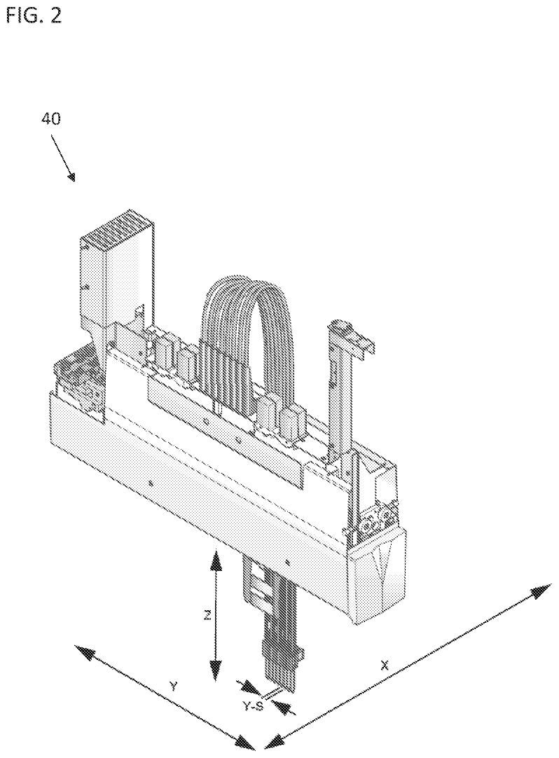

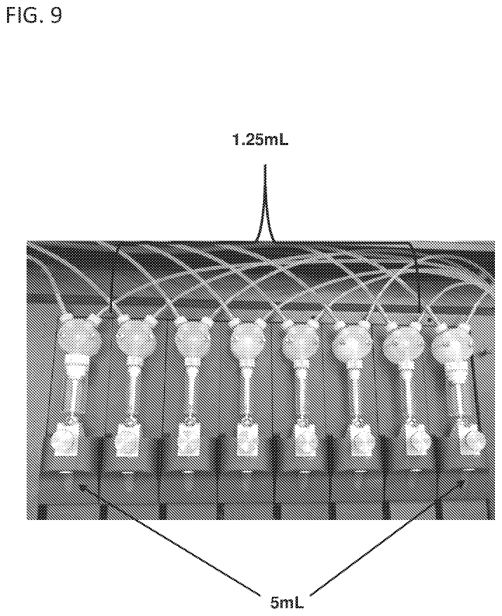

In embodiments, the liquid handling system 30 includes a flexible channel liquid manipulation module 40, such as a liquid displacement flexible channel arm (see, for example, FIG. 2). In embodiments, a flexible channel liquid manipulation module 40 is configured to transfer liquid, such as a liquid containing mammalian cells, from one container to another, for example flat or round bottom plates, tubes, such as conical tubes, and the like. This arm is termed “flexible” because each pipetting channel may operate independently and module 40 may therefore be capable of transferring liquids of different volumes simultaneously. In embodiments, the flexible liquid manipulation module 40 is multiplex in that it has multiple separate pipetting channels that separately manipulate samples, such as different samples of liquid containing mammalian cells, such as T cells, (e.g., CD4+ T cells and/or CD8+ T cells). In certain embodiments, the channels, or a subset of channels, can operate independently. For example, the control system 20 can be programmed to operate the channels, or a subset of the channels independently. In embodiments, each pipetting channel operates independently and the flexible liquid manipulation module 40 is capable of transferring liquids of different volumes simultaneously. In embodiments, the flexible liquid manipulation module 40 has between about 2 and 196 or more channels. In embodiments, the flexible liquid manipulation module 40 uses liquid displacement technology for liquid transfer. In embodiments, liquid transfer is performed by pressure differences created by diluter syringe pistons. For example, a downward piston movement creates a negative pressure difference and enables the aspiration of liquid at the pipette tip ends, while an upward piston movement creates a positive pressure difference and enables the dispensing of liquid out of the pipette tips. In certain embodiments, the tip of the pipette(s) (which would be the portion of the pipette in contact with the liquid) uses a disposable tip (DiTi) to provide for a sterile culture. In other embodiments, the tips are fixed but sterilized, for example with UV light, or other chemical or radiation treatment. The DiTi configuration enables the use of syringes between about 0.5 mL and about 5 mL, such as between about 1.25 mL and about 5 mL, for example 1.25 mL syringes and 5 mL syringes. In embodiments, the individual pipette channels are able to accurately manipulate fluid volumes between about 0.5-5000 μL (see, Liquid Class Determination section below). DiTi types can also be interchanged directly within a cell culture method, such as described below with respect to T cell culture. In specific embodiments, the liquid handling system 30 uses a liquid displacement flexible channel arm (FCA) (see, for example, FIG. 2). In the implementation shown in FIG. 2 the liquid FCA is an eight pipetting channel system that utilizes liquid displacement technology for liquid transfer. A standard FCA syringe configuration includes 8×1.25 mL syringes, which allow for the transfer of up to 1 mL of liquid. Because of the requirements for large volume transfers for cell culture media, the syringe configuration for the system was developed to allow for 5 mL syringes (see FIG. 9). With reference to FIG. 9, the 5 mL syringes were placed in positions 1 and 8 to limit steric hindrance towards the remaining 6 1.25 mL syringes. Additionally, having consecutive 1.25 mL syringes is highly beneficial for scale down method scripting. Overall, the 1.25/5 mL syringe configuration enables large volume transfers, but provides flexibility for method scripting and development.

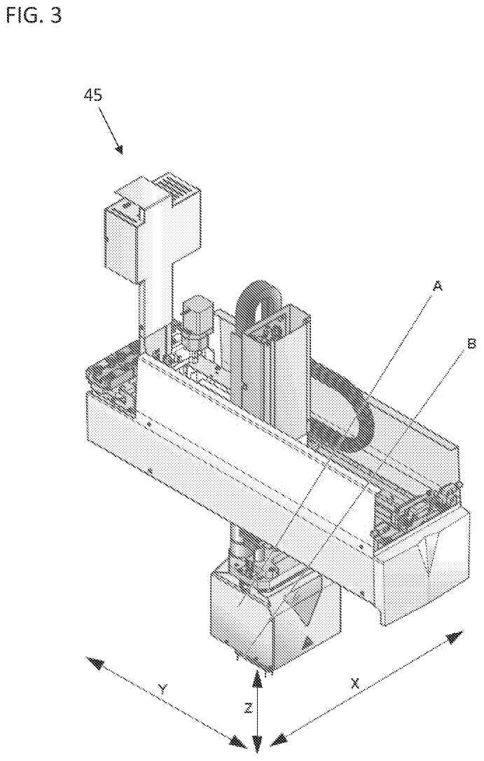

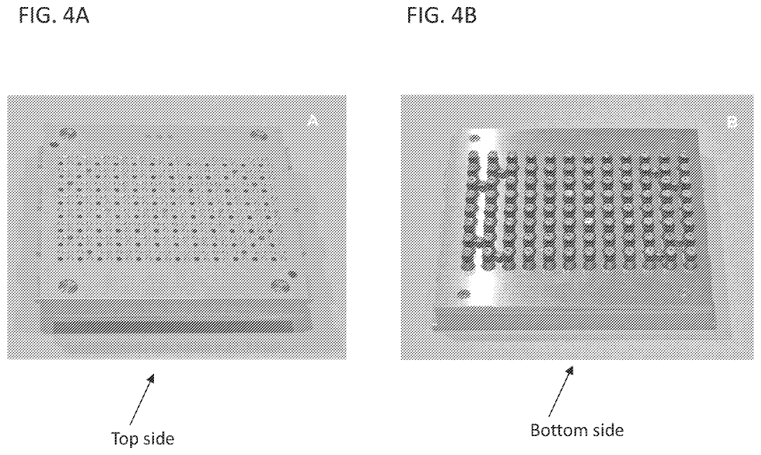

In embodiments, the liquid handling system 30 optionally includes a static multichannel liquid manipulation module 45, such as a multiple channel arm (MCA) (see FIG. 3) in addition to the flexible channel liquid manipulation module 40. The static multichannel liquid manipulation module 45 is “static” in that it is unable to differentially transfer liquids of different volumes simultaneously. The static multichannel liquid manipulation module 45 can be used when the volume of liquid is the same across the channels. Typically, the static multichannel liquid manipulation module 45 is used for the transfer of liquid in a multiwell format, such as a 96 or 384 channel format. In embodiments, the static multichannel liquid manipulation module 45 is an air displacement system, having 384 plungers that perform aspiration and dispensing steps based on a pressure difference within each cylinder. However, unlike the flexible channel liquid manipulation module 40, the static multichannel liquid manipulation module 45 plungers move concurrently and are therefore unable to differentially transfer liquids of different volumes simultaneously. The static multichannel liquid manipulation module 45 may be compatible with multiple adapter types. In certain examples of the system, a 96 channel adapter is used in conjunction with the static multichannel liquid manipulation module 45 (see FIGS. 4A and 4B). In certain embodiments, the tip of the pipettes (which would be the portion of the pipette in contact with the liquid) uses a disposable tip (DiTi) to provide for a sterile culture. In other embodiments, the tips are fixed but sterilized, for example with UV light, or other chemical or radiation treatment. By way of example, DiTi tips are picked up and liquid is transferred with either all 96 DiTis together, the first 2 rows of 12 DiTis or first four columns of DiTis, depending on the system 10 configuration. In embodiments, the 96 channel adapter uses DiTis (as oppose to fixed tips), and enables the multiplex transfer of liquid between 0.2 μL and 250 μL, such as between 0.5 μL and 125 μL.

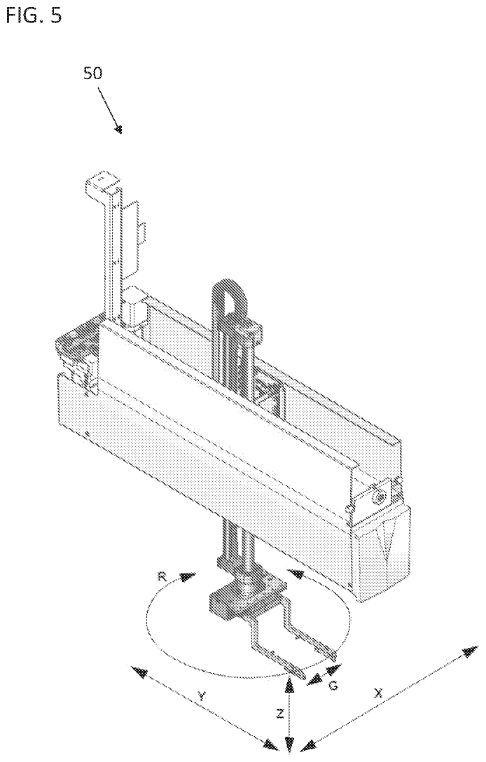

In embodiments, the liquid handling system 30 may include a container manipulation module 50, such as a long z-axis robotic gripper arm (RGA) (see FIG. 5). The container manipulation module 50 can be fitted with different gripper configurations or heads to manipulate different shapes and sizes of containers depending on the activity. In certain embodiments, the container manipulation module 50 is used in a sterile environment, for example for sterile cell culture work as described above. In certain embodiments, the different gripper configurations or heads may be automatically changed by the system 10, for example under the control of the control system 20. Based on the gripper configuration, the container manipulation module 50 allows for the transport of a gamut of labware throughout a worktable 60 and underneath. The labware may include microplates, deepwell plates, conical tubes, DiTi boxes and the like. The container manipulation module 50 may also be used for the transport of labware to and from storage positions and devices. In certain embodiments, such as with a long z-axis robotic gripper arm (RGA), the container manipulation module 50 moves containers in the x, y, and z direction. As depicted in FIG. 5 gripper fingers of the container manipulation module 50 can also open and close (G), as well as rotate 360° (R).

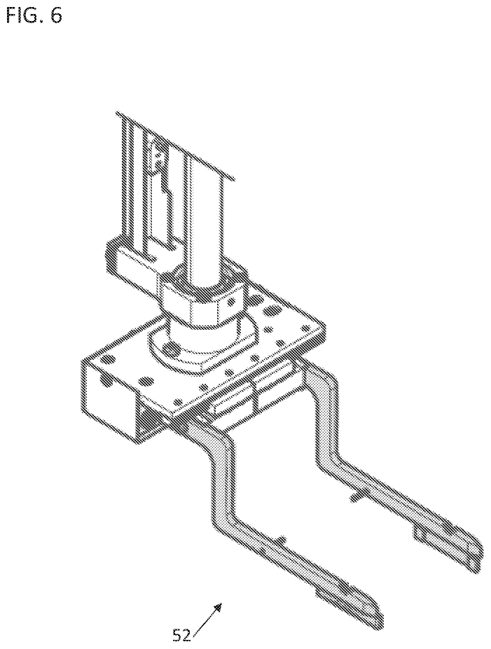

In certain configurations, the container manipulation module 50 uses eccentric fingers 52 (see, for example, FIG. 6). Eccentric fingers 52 allow for horizontal access and transport of labware. Eccentric fingers 52 enable the transport of standard cell culture (e.g., 6 well and 24 well plates) and sampling plates (e.g., 1.0 mL deep well plates, 96 flat/round plates). The eccentric fingers 52 further allow for hotel 105 access and loading (discussed below).

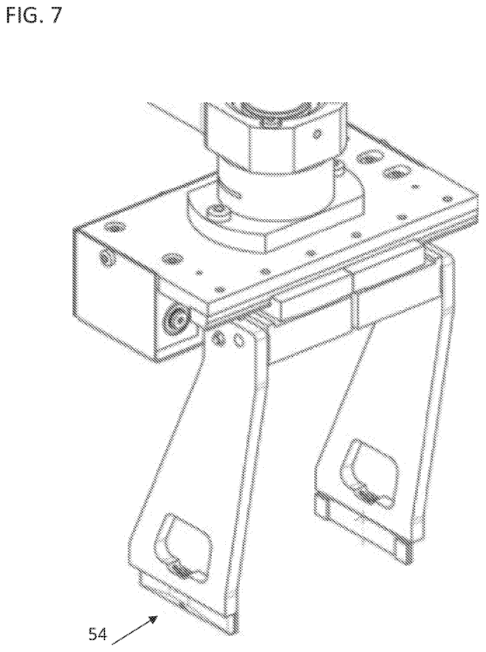

In certain configurations, the container manipulation module 50 uses centric fingers 54 (see for example, FIG. 7). Centric fingers 54 have vertical access to labware, and are used to accessing sites where horizontal access is limited. The centric fingers 54 allow for the transport of all deepwell cell culture plates (centrifuge and expansion plates), and centrifuge components around the worktable 60 and below.

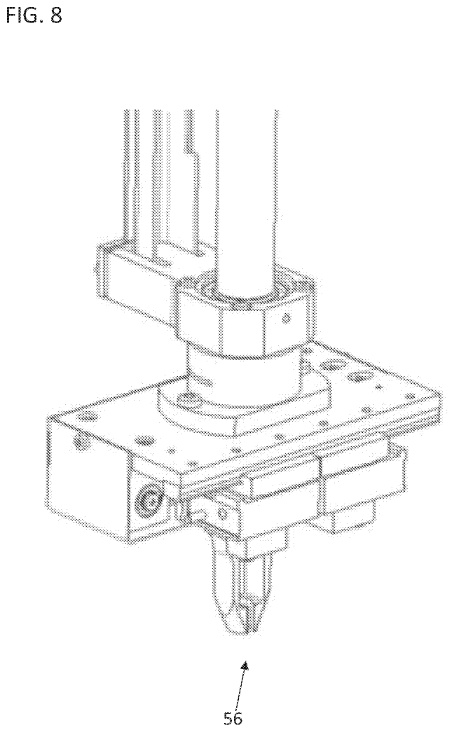

In certain configurations, the container manipulation module 50, uses tube fingers 56 (see FIG. 8). The tube fingers 56 are used for the transport of tube type labware. The tube fingers 56 may also be used for the capping and decapping of cryovials (see activation and harvest unit operations) and 50 mL conical tubes (see activation unit operation).

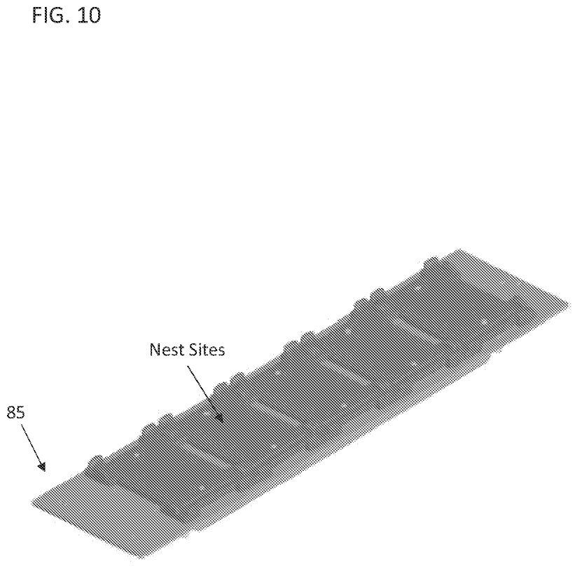

Referring again to FIG. 1, in addition to the modules discussed above, the liquid handling system 30 includes a worktable 60 with components configured to enable the current scale down applications set forth in the methods below. These applications include activation, transduction, inoculation, expansion, debeading and harvest unit operations for T cells. Deck segments 85, nest types (refer to FIG. 10), trough runners, tube runners, hotels 105, custom labware and integrated devices are configured to enable maximal processing of each unit operation without significant worktable 60 modifications between different unit operations. The worktable 60 layout per unit operation method uses nest sites and hotels 105 to maximize the amount of labware used and thereby, maximize the number of conditions performed with each unit operation greatly enhancing the multiplex ability of the system 10.

In embodiments, deck segments 85 are deck components that can be positioned on the worktable 60 according to the configuration of the instrument user (see, for example, FIG. 10). Deck segments 85 house nest sites, which are utilized to hold labware. In embodiments, the worktable 60 is decorated with 25×7 mm nests to enable the use of both microplate and deepwell plates.

In embodiments, the liquid handling system 30 includes trough runners 90, such as 320 mL reagent trough runners. The 320 mL reagent trough runners are 2-position grid segments that hold 3×320 mL reagent troughs. 320 mL troughs hold up to 256 mL of liquid, and may be chosen to hold large volume reagents such as cell culture media.

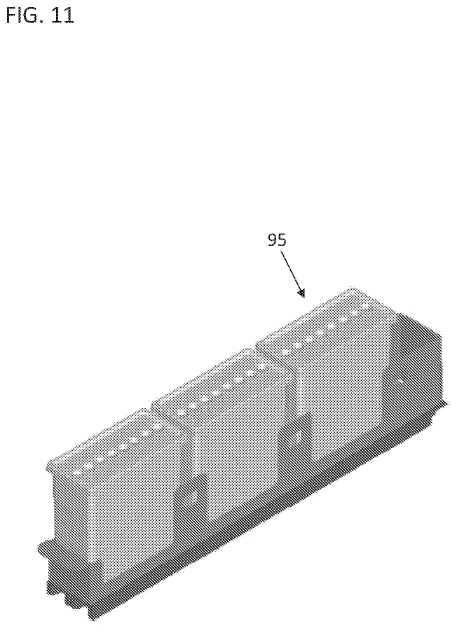

In embodiments, the liquid handling system 30 includes reagent troughs 95, such as 100 mL reagent troughs (see FIG. 11). 100 mL reagent troughs holds 3×100 mL reagent troughs. 100 mL hold up to 80 mL of liquid, and were chosen to hold large volume reagents such as cryopreservation media and cell viability measurement reagents, for example Guava Viacount reagent.

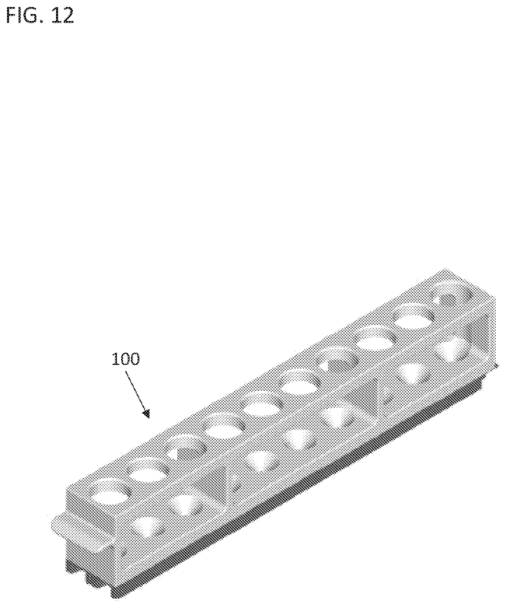

In embodiments, the liquid handling system 30 includes conical tube runners 100, such as 50 mL conical tube runners (see FIG. 12). In an example, the 50 mL conical tube runners are 2-position grid segments that holds 10×50 mL conical tubes. Within the automated scale down methods, 50 mL conical tubes are used for large volume cell mixtures, namely in the activation unit operation, whereby cells are washed with fresh cell culture media and centrifuged.

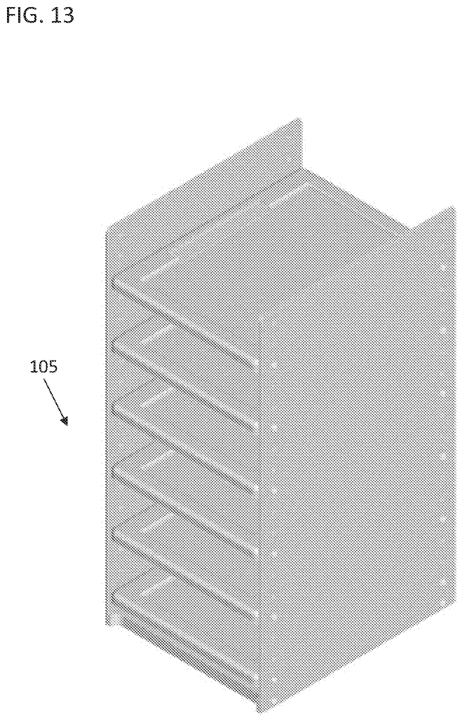

In embodiments, the liquid handling system 30 includes hotels 105 (see FIG. 13). Hotels 105 are utilized for the storage of plate type labware. In embodiments, hotels 105 have between 2 and 10 positions. Multiple hotels 105 can be used to increase the number of positions. These allow for the maximization of the worktable 60 space. Labware can be stored in hotels 105 until use and can then be transferred to the worktable 60 when needed via container manipulation module 50 eccentric fingers 52. In certain embodiments, six position hotels 105 are chosen due to their ability to hold a diverse set of labware. Within the automated scale down process, one or more of 24-flat well plates, 6-well plates, 96-deepwell plates, 96-flat microplates, 96-round microplates, 6 mm lids, 9 mm plate lids, metal expansion lids, etc., may be stored within the 6 position hotels 105, for unit operation. It may be understood that additional or alternative labware may be stored within hotels 105, without departing from the scope of this disclosure.

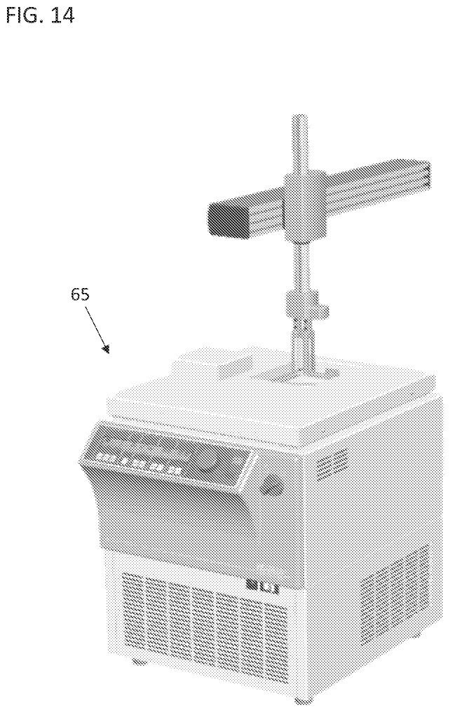

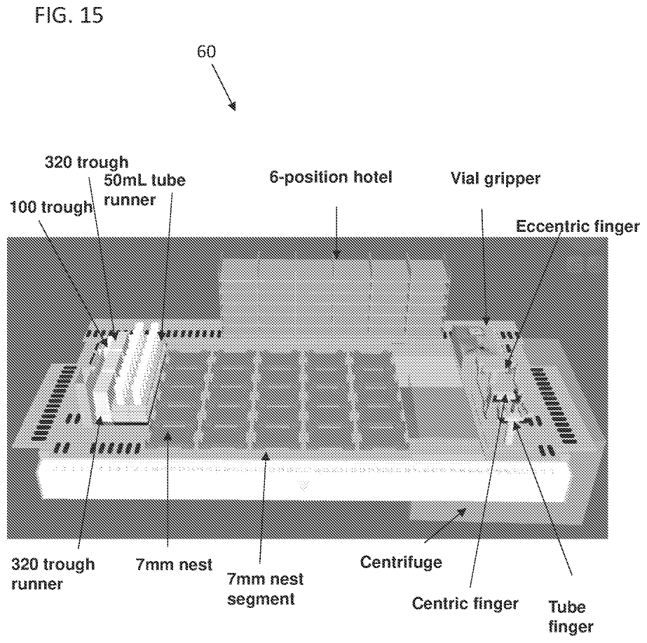

In embodiments, the liquid handling system 30 includes a robotic centrifuge 65 (see, for example, FIG. 14). In certain embodiments, the robotic centrifuge 65 is temperature controlled. In certain embodiment, the robotic centrifuge 65 is computer controlled, for example with the control system 20 to sense and control rotor positioning, for example to allow for tubes and or plates to be easily manipulated, placed in, and/or removed from robotic centrifuge 65. In certain embodiments, the robotic centrifuge 65 has between about 2 and about 8 positions for the insertion of one or more containers to provide flexibility. In certain embodiments, the robotic centrifuge 65 is a four (4) position robotic centrifuge that is temperature controlled with computer controlled rotor positioning. In certain embodiments, the robotic centrifuge 65 has below deck capabilities (e.g. vertical vs horizontal access). In the automated T cell culture methods disclosed below, the container manipulation module 50, such as a long z-axis robotic gripper arm (RGA), picks up labware by centric fingers 54, then transfers the labware vertically into the robotic centrifuge 65 via a top loading automated door. These manipulations, including operation of the robotic centrifuge 65 can be controlled by the control system 20, for example based on user inputs, or a preexisting program file with instructions for operating the robotic centrifuge 65 and the container manipulation module 50.

In embodiments, the liquid handling system 30 includes a vial gripper module 70). In embodiments, the vial gripper module 70 is a pneumatic device that enables the capping and decapping of tubes, such as conical tubes. This may include standard 15 mL and 50 mL tube sizes. Conical tube capping/decapping is done for tube centrifugation steps, such as the method disclosed below. Using the tube fingers 56, the container manipulation module 50 transfers conical tubes into the vial gripper module 70, and based on a pressure change, the vial gripper module 70 grips the tubes. The container manipulation module 50 then picks up the tube's cap and places it onto the conical tube and caps the tube. Lastly, the container manipulation module 50 transfers the tubes into a centrifuge tube adapter for centrifugation in the robotic centrifuge 65. Post centrifugation, the tube is returned back to the vial gripper module 70 for decapping. In the methods below, the vial gripper module 70 is used for tube centrifugation steps in the activation unit process. These manipulations, including operation of the vial gripper module 70 can be controlled by the control system 20, for example based on user inputs, or a preexisting program file with instructions for operating the vial gripper module 70 and robotic centrifuge 65 and the container manipulation module 50.

In embodiments, the liquid handling system 30 optionally includes a cell counting module 75, for example to remove manual cell viability determination. In certain embodiments, the container manipulation module 50 directly transfers a counting plate into the cell counting module 75. In certain embodiments, the cell counting module 75 transfers the viable cell count (VCC) measurements to the control system 20. The automated VCC measurements allow for direct propagation of the methods, without operator interaction.

In embodiments, the liquid handling system 30 includes a portable cryovial cooling chamber/cap holder 80. In embodiments, the cryovial cooling chamber is a 12 position holder for 2 mL cryovials. In embodiments cryovial cooling chamber/cap holder 80 is threaded to allow for capping and decapping functions with the container manipulation module 50 tube fingers 56. The cryovial cooling chamber/cap holder 80 can be placed in a freezer prior to use and will keep cryovials at the source temperature for the duration of the method. The cryovial cooling chamber/cap holder 80 may be portable to allow possible transport to a temperature controlled centrifuge during holding or pausing steps. The cap holder may be a custom unit and may be used to store 2 mL cryovial caps for capping and decapping steps.

In embodiments, the liquid handling system 30 includes portable tube centrifuge adapters 110, such as 50 mL tube centrifuge adapters 110. The 50 mL tube centrifuge adapters 110 are custom centrifuge buckets that enable the centrifugation of 50 mL tubes. These adapters are also used as tube holders for steps that require tube manipulation. The 50 mL tube centrifuge adapters 110 were created to be portable to allow easy transport in and out of the centrifuge with the container manipulation module 50. On the worktable 60, they are placed on a custom centrifuge adapter nest.

In embodiments, the liquid handling system 30 includes a tube cap holder 115, such as a 50 mL tube cap holder. The 50 mL tube cap holder is a custom unit that is used to store 50 mL tube caps for capping and decapping steps.

The disclosed systems, as well as the implementation of custom carriers, enables a fully automated T cell culture platform. The implementation of this platform allows for more consistent experimentation, thereby decreasing operator-based variability introduced in experiments. Compared to a human operator, this platform will improve the number of experiments performed and the time required per experiment.

The control system 20 as shown in may include one or more computing devices. In embodiments, a computing device includes a number of components, such as one or more processors and at least one communication module. In various embodiments, the one or more processors each include one or more processor cores. In various embodiments, the at least one communication module is physically and/or electrically coupled to the one or more processors. In further implementations, the communication module is part of the one or more processors. In various embodiments, the computing device includes a printed circuit board (PCB). For these embodiments, the one or more processors and communication module is disposed thereon. Depending on its applications, the computing device includes other components that may or may not be physically and electrically coupled to the PCB. These other components include, but are not limited to, a memory controller, volatile memory (e.g., dynamic random access memory (DRAM) (not shown)), non-volatile memory such as read only memory (ROM), flash memory, an I/O port, a digital signal processor, a crypto processor, a graphics processor, one or more antenna, a display (e.g., touch-screen display), a display controller (e.g., touch-screen display controller), a battery, an audio codec, a video codec, and a mass storage device (such as hard disk drive, a solid state drive, compact disk (CD), digital versatile disk (DVD)), and so forth.

In some embodiments, the one or more processors is/are operatively coupled to system memory through one or more links (e.g., interconnects, buses, etc). In embodiments, system memory is capable of storing information that the one or more processors utilizes to operate and execute programs and operating systems. In different embodiments, system memory is any usable type of readable and writeable memory such as a form of dynamic random access memory (DRAM). In embodiments, the computing device includes or is otherwise associated with various input and output/feedback devices to enable user interaction with the computing device and/or peripheral components or devices associated with the computing device by way of one or more user interfaces or peripheral component interfaces. In embodiments, the user interfaces may include, but are not limited to, a physical keyboard or keypad, a touchpad, a display device (touchscreen or non-touchscreen), speakers, microphones, image sensors, haptic feedback devices and/or one or more actuators, and the like. In some embodiments, the computing device can comprise a memory element (not shown), which can exist within a removable smart chip or a secure digital (“SD”) card or which can be embedded within a fixed chip on the dental ex. In certain example embodiments, Subscriber Identity Component (“SIM”) cards may be used. In various embodiments, the memory element may allow a software application resident on the device.

In some embodiments, the one or more processors, flash memory, and/or a storage device includes associated firmware storing programming instructions configured to enable the computing device, in response to execution of the programming instructions by one or more processors, to practice all or selected aspects of a methods disclosed herein, in accordance with embodiments of the present disclosure.

In embodiments, the communication module enables wired and/or wireless communications for the transfer of data to and from the computing device, such as too and from the liquid manipulation system 30 and/or the various modules thereof. In various embodiments, the computing device also includes a network interface configured to connect the computing device to one or more networked computing devices wirelessly via a transmitter and a receiver (or optionally a transceiver) and/or via a wired connection using a communications port. In embodiments, the network interface and the transmitter/receiver and/or communications port are collectively referred to as a “communication module”. In embodiments, the wireless transmitter/receiver and/or transceiver may be configured to operate in accordance with one or more wireless communications standards. The term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In embodiments, the computing device includes a wireless communication module for transmitting to and receiving data, for example for transmitting and receiving data from a network, such as a telecommunications network. In embodiments, the computing device is directly connect with one or more devices via the direct wireless connection by using, for example, Bluetooth and/or BLE protocols, WiFi protocols, Infrared Data Association (IrDA) protocols, ANT and/or ANT+ protocols, LTE ProSe standards, and the like. In embodiments, the communications port is configured to operate in accordance with one or more known wired communications protocol, such as a serial communications protocol (e.g., the Universal Serial Bus (USB), FireWire, Serial Digital Interface (SDI), and/or other like serial communications protocols), a parallel communications protocol (e.g., IEEE 1284, Computer Automated Measurement And Control (CAMAC), and/or other like parallel communications protocols), and/or a network communications protocol (e.g., Ethernet, token ring, Fiber Distributed Data Interface (FDDI), and/or other like network communications protocols).

In embodiments, the computing device is configured to run, execute, or otherwise operate one or more applications. In embodiments, the applications include native applications, web applications, and hybrid applications. In embodiments, native applications are platform or operating system (OS) specific or non-specific. In embodiments, native applications are developed for a specific platform using platform-specific development tools, programming languages, and the like. Such platform-specific development tools and/or programming languages are provided by a platform vendor. In embodiments, native applications are pre-installed on computing device during manufacturing, or provided to the computing device by an application server via a network. Web applications are applications that load into a web browser of the computing device in response to requesting the web application from a service provider. In embodiments, the web applications are websites that are designed or customized to run on a computing device by taking into account various computing device parameters, such as resource availability, display size, touch-screen input, and the like. In this way, web applications may provide an experience that is similar to a native application within a web browser. Web applications may be any server-side application that is developed with any server-side development tools and/or programming languages, such as PHP, Node.js, ASP.NET, and/or any other like technology that renders HTML. Hybrid applications may be a hybrid between native applications and web applications. Hybrid applications may be a standalone, skeletons, or other like application containers that may load a website within the application container. Hybrid applications may be written using website development tools and/or programming languages, such as HTML5, CSS, JavaScript, and the like. In embodiments, hybrid applications use browser engine of the computing device, without using a web browser of the computing device, to render a website's services locally. In some embodiments, hybrid applications also access computing device capabilities that are not accessible in web applications, such as the accelerometer, camera, local storage, and the like.

Any combination of one or more computer usable or computer readable medium(s) may be utilized with the embodiments disclosed herein. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. Note that the computer-usable or computer-readable medium can even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, RF, etc.

Computer program code for carrying out operations of the present disclosure may be written in any combination of one or more programming languages, including an object oriented programming language such as Java, Smalltalk, C++ or the like and conventional procedural programming languages, such as the “C” programming language or similar programming languages or a programming language native to the control system 20. The program code may execute entirely on the user's computing device, partly on the user's computing device, as a stand-alone software package, partly on the user's computing device and partly on a remote computer or entirely on the remote computer or server. In the latter scenario, the remote computer may be connected to the user's computing device, through any type of network, including a local area network (LAN) or a wide area network (WAN), or the connection may be made to an external computing device, (for example, through the Internet using an Internet Service Provider), or wireless network, such as described above.

Furthermore, example embodiments may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium. A code segment may represent a procedure, a function, a subprogram, a program, a routine, a subroutine, a module, program code, a software package, a class, or any combination of instructions, data structures, program statements, and the like.

In various embodiments, an article of manufacture may be employed to implement one or more methods as disclosed herein. The article of manufacture may include a computer-readable non-transitory storage medium and a storage medium. The storage medium may include programming instructions configured to cause an apparatus to practice some or all aspects of the methods disclosed herein, in accordance with embodiments of the present disclosure.

The storage medium may represent a broad range of persistent storage medium known in the art, including but not limited to flash memory, optical disks or magnetic disks. The programming instructions, in particular, may enable an apparatus, in response to their execution by the apparatus, to perform various operations described herein. For example, the storage medium may include programming instructions configured to cause an apparatus to practice some or all aspects of a method herein, in accordance with embodiments of the present disclosure.

Exemplary Process

While the system described above can be used to perform the method described below, it should in no way be construed as limiting the systems that can be used for the methods and units disclosed herein.

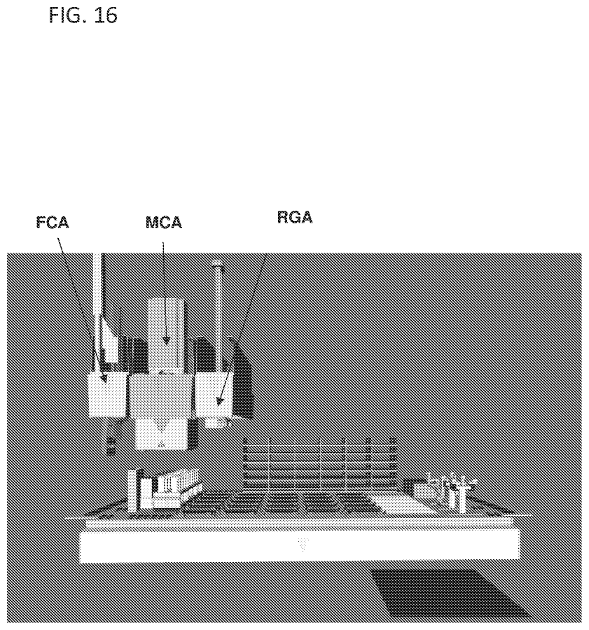

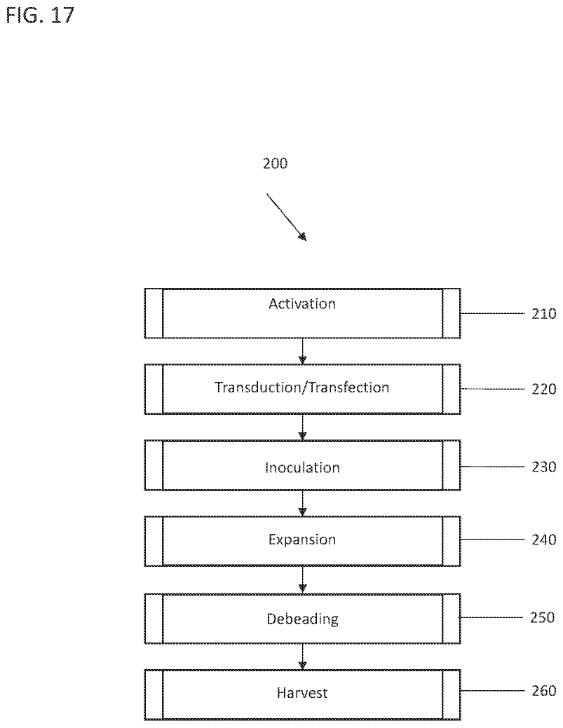

With reference to FIG. 17, the disclosed method 200 includes six units that independently encompass activation unit operation 210, transduction unit operation 220, inoculation unit operation 230, expansion unit operation 240, debeading unit operation 250, and expansion unit operation 260 for T cells. Working embodiments of the methods disclosed herein have been implemented on a liquid handling system (for example, a highly modified Tecan Fluent 780 system). Working embodiments, of the disclosed methods were performed using a FCA, MCA, and RGA, as discussed above (see FIG. 16). These arms allow for liquid and labware transfer, respectively. It is noted that activation unit operation 210, transduction unit operation 220, inoculation unit operation 230, expansion unit operation 240, debeading unit operation 250, and harvest unit operation 260 can be performed for several experimental setups simultaneously. For example, the times that cells are in the mammalian cell incubator may be staggered or interleaved with the times other cells are being manipulated by the remaining components of the system 10.

Samples

The methods and systems provided herein are used with mammalian cells, such as cells isolated from a subject, with particular relevance to T cells, for example CD4+ and/or CD8+ T cells. In some embodiments, the systems and methods disclosed herein use cells or compositions thereof isolated from biological samples, such as those obtained from or derived from a subject, such as one having a particular disease or condition or in need of a cell therapy or to which cell therapy will be administered. In some embodiments, the systems and methods disclosed herein use cells or compositions thereof isolated from biological samples, such as those obtained from or derived from a subject, such as a healthy donor. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom. In some embodiments, the systems and methods disclosed herein are used with non-primary cells, such as cell lines, for example as part of a testing procedure, or validation of methodology and the like.

In embodiments, a sample is blood or a blood-derived sample, or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources. In some examples, the cells are obtained from the circulating blood of a subject, such as by apheresis or leukapheresis. The samples, in some aspects, contain leukocytes, including T cells, monocytes, granulocytes, B cells, red blood cells, and/or platelets, and in some aspects contains cells other than red blood cells and platelets. In certain embodiments, the cells for use in the system and methods disclosed herein are T cells enriched for CD4+ T cells. In certain embodiments, the cells for use in the system and methods disclosed herein are T cells enriched for CD8+ T cells. In some embodiments, two separate compositions of enriched CD4+ T cells and enriched CD8+ T cells are separately subjected to the various systems and methods disclosed herein. In some embodiments, the single composition is a composition of enriched CD4+ and CD8+ T cells, for example cells that have been separately enriched and that have been combined from separate compositions. Methods of enriching for CD4+ T cells and/or CD8+ T cells are known in the art.

Activation Unit Operation

The activation unit operation 210 begins with worktable set up and with reference to the system 10 of FIG. 1, the control system 20 prompts the user/operator to set up the worktable 60, for example with DiTi, reagent troughs, cell culture media, cell counting reagent, etc. The control system 20 further prompts the user to input experimental parameters, such as the number of T cell donors, the number of activation agents and the number of conditions run. Other analytics parameters can be set by the user. The user can be prompted to enter these parameters in real time, or as part of a script, for example, set up by the user prior to initiation of the system 10 of the method 200 as shown in FIG. 17. In embodiments, the user is prompted to input the number of conditions to be run. In embodiments, the user is prompted to input the desired sampling volume. In embodiments, the user is prompted to input whether to perform sampling at the end of the method. If the user selects yes, the user is then prompted to specify the total cellular material and AAA/flow cytometry sampling volumes. In embodiments, if multiple donors are selected the user is then prompted to enter the number of CD4 and CD8 donors, as well as the total number of cryovials required for the method. In embodiments, the user is prompted to also include the activation reagent volume to be dispensed per well. By reference to “activation reagent” herein, it is meant one or more agents.

Once the experimental inputs have been received by the control system 20, the control system determines the worktable 60 configuration and labware needed for the selected parameters. In some embodiments, in this and the other unit processes, the labware is automatically placed in the automated liquid handling system 30 for example using the container manipulation module 50, for example, under direction from the control system 20. In other embodiments, some or all of the labware is placed on the worktable 60 by one or more users for example as prompted by the control system 20. In certain embodiments, a number of 50 mL conical tubes (or other relevant tube type/volume) are placed onto the worktable 60 according to the number of donors input. In embodiments, the conical tubes are placed in the centrifuge tube adapter. In embodiments, a user is prompted by the control system to place 50 mLconical tubes onto the worktable 60 according to the number of donors input. In embodiments, the container manipulation module 50, such as an RGA, with tube fingers 56, transfers 50 mLconical tubes to the vial gripper module 70 and decaps them. The tube caps are placed onto conical tube cap holders and the tube is returned to the centrifuge tube adapter after centrifugation.

In certain embodiments, an “n” number of cellular material plates based on the number of conditions input is placed on the worktable 60. Sampling plates may include a 96-deepwell plate, a 96-well low attachment plate (cell counting), and a 96-well round bottom plate (AAA/flow cytometry). In certain embodiments, a user is prompted to setup the worktable 60 with sampling and “n” number of cellular material plates based on the number of conditions input. In embodiments, the container manipulation module 50, such as an RGA, using eccentric fingers 52, places all cellular material and sampling plates into hotels 105.

Once worktable setup is complete, washing is initiated by the control system 20. A number of cryovials per donor for both CD4+ and CD8+ samples may be selected. In embodiments, the number of cryovials may be determined by the control system 20. In certain embodiments, a user is prompted to select the number of cryovials per donor for both CD4+ and CD8+ samples. Using the inputs of number of CD4+ and CD8+, number of cryovials needed from the worktable set up, and the number of number of cryovials per donor for both CD4+ and CD8+ samples, the flexible liquid manipulation module 40, such as an FCA, transfers the contents in the cryovial to the 50 mL conical tubes. In embodiments, the flexible liquid manipulation module 40 dispenses balance cell culture media to reach a selected volume for each 50 mL conical tube. In embodiments, the container manipulation module 50, using tube fingers 56, transfers the 50 mL conical tubes to the vial gripper module 70 and re-caps each tube. In embodiments, the container manipulation module 50, using tube fingers 56, transfers the 50 mL conical tubes back into the centrifuge tube adapter. In embodiments, the container manipulation module 50 replaces the tube fingers 56 with centric fingers 54, and transports the centrifuge tube adapters with tubes vertically into the robotic centrifuge 65 for centrifugation.

Post centrifugation, the centrifuge adapter, along with the conical tubes are returned to the worktable 60. In embodiments, the container manipulation module 50, replaces the centric fingers 52 with tube fingers 56, and transfers the tubes to the vial gripper module 70 and decaps them. In embodiments, the flexible liquid manipulation module 40, such as FCA then removes the supernatant without disrupting the cell pellet for each conical tube. In embodiments, each tube is then resuspended based on the VCC as selected and the number of cryovials added.

Once the washing is complete, sampling is initiated by the control system 20. Each 50 mL conical tube is mixed, then a total sample volume is aspirated per tube and dispensed into the 96-deepwell plate. The dispensed sampling volume is then mixed, and aliquoted into low attachment cell counting plates. A cell counting reagent is then dispensed into the low attachment cell counting plates according the number of conditions input. In certain embodiments, the cell counts for the sampling plates are automatically read by the cell counting module 75. In other embodiments, the sampling plates are brought to the front of the worktable 60 for user reachability, then removed from the worktable 60 by a user for manual cell counting. Cell concentration measurements are obtained by the system controller 20, either automatically from the cell counting module 75 or as manually entered by a user. Based on the current VCC, the required cell volume to reach the target VCC is calculated.

Once sampling is complete, activation is initiated by the control system 20. Activation reagent is added to the worktable 60, for example automatically. In certain embodiments, a user is prompted to add the activation reagent to the worktable 60. In embodiments, the container manipulation module 50, such as an RGA, using eccentric fingers 52, places “n” number of welled plates (for example, a 6-well, a 12-well, a 24-well plate, and/or a 48-well plate, etc.) onto the worktable 60 from hotels 105 according the condition number. The welled plates may be round-bottom plates, flat-bottom plates, etc. For a six well plate, one plate is required per every six conditions. In embodiments, the activation reagent is then dispensed onto each well of the plate according to the number of conditions input. In embodiments, the flexible liquid manipulation module 40, then proceeds by mixing each tube. The flexible liquid manipulation module 40 then dispenses the required cell volume to reach the total nucleated cell count (TNC) according to the user input. The flexible liquid manipulation module 40 follows by dispensing balance cell culture media into each well of the plate per condition to reach the desired VCC.

Once activation is complete, optionally sampling is initiated by the control system 20. If sampling is desired, each sample well is mixed, then a total sample volume is aspirated per sample and dispensed into the 96-deepwell plate. The dispensed sampling volume is then mixed, and aliquoted into the cell counting and AAA/flow cytometry plates. In certain embodiments, the cell counts for the sampling plates are automatically read by the cell counting module 75. In other embodiments, the sampling plates are then brought to the front of the worktable 60 for user reachability, then removed from the worktable 60 by a user for manual cell counting. Cell concentration measurements are obtained by the system controller 20, either automatically from the cell counting module 75 or as manually entered by a user. In embodiments, the plates are automatically relidded. In embodiments, the plates are automatically transferred to an incubator. In embodiments, the remaining labware is automatically removed from the worktable. In embodiments, a user is prompted to place the plates in an incubator. In embodiments, a user is prompted remove all remaining labware from the worktable.

If sampling is not done, no sampling is initiated by the control system 20. In embodiments, the plates are automatically relidded. In embodiments, the plates are automatically transferred to an incubator. In embodiments, all remaining labware is automatically removed from the worktable. In embodiments, a user is prompted to place plates in an incubator. In embodiments, a user is prompted to remove all remaining labware from the worktable.

In some embodiments, the provided methods and systems are used in connection with incubating cells under activation conditions, for example with one or more reagents added during the activation unit operation 220. In some embodiments, the activation conditions include conditions that activate or stimulate, and/or are capable of activating or stimulating a signal in the cell, e.g., a CD4+ T cell or CD8+ T cell. In some embodiments, the activation conditions include one or more steps of culturing, cultivating, incubating, activating, propagating the cells with and/or in the presence of an activation reagent, e.g., a reagent that activates or stimulates, and/or is capable of activating or stimulating a signal in the cell.

In some embodiments, the incubation under activation conditions can include culture, cultivation, stimulation, activation, propagation, including by incubation in the presence of activation conditions, for example, conditions designed to induce proliferation, expansion, activation, and/or survival of cells in the population, to mimic antigen exposure, and/or to prime the cells for transduction, such as for the introduction of a recombinant antigen receptor. In particular embodiments, the activation conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In particular embodiments, the activation conditions include incubating, culturing, and/or cultivating the cells with an activation reagent. In certain embodiments, the activation reagent contains or includes a bead. In certain embodiments, the start and or initiation of the incubation, culturing, and/or cultivating cells under activation conditions occurs when the cells come into contact with and/or are incubated with the activation reagent. In particular embodiments, the cells are incubated with the activation reagent prior to, during, and/or subsequent to transducing the cells, e.g., introducing a recombinant polynucleotide into the cell such as by transduction or transfection.

In some embodiments, the composition of enriched T cells are incubated at a ratio of activation reagent and/or beads to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of activation reagent and/or beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of activation reagent to cells is about 1:1 or is 1:1.

In particular embodiments, an activation reagent includes one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes IL-2.

In particular embodiments, an activation reagent includes IL-2, e.g., recombinant IL-2. Without wishing to be bound by theory, particular embodiments contemplate that CD4+ T cells that are obtained from some subjects do not produce, or do not sufficiently produce, IL-2 in amounts that allow for growth, division, and expansion throughout the process for generating a composition of output cells, e.g., engineered cells suitable for use in cell therapy. In some embodiments, incubating a composition of enriched CD4+ T cells under activation conditions in the presence of recombinant IL-2 increases the probability or likelihood that the CD4+ T cells of the composition will continue to survive, grow, expand, and/or activate during the incubation step and throughout the process.

In certain embodiments, the amount or concentration of the one or more cytokines are measured and/or quantified with International Units (IU). International units may be used to quantify vitamins, hormones, cytokines, vaccines, blood products, and similar biologically active substances. In some embodiments, IU are or include units of measure of the potency of biological preparations by comparison to an international reference standard of a specific weight and strength (e.g., WHO 1st International Standard for Human IL-2, 86/504). International Units are the only recognized and standardized method to report biological activity units that are published and are derived from an international collaborative research effort. In particular embodiments, the IU for composition, sample, or source of a cytokine may be obtained through product comparison testing with an analogous WHO standard product. For example, in some embodiments, the IU/mL of a composition, sample, or source of human recombinant IL-2, IL-7, or IL-15 is compared to the WHO standard IL-2 product (NIBSC code: 86/500), the WHO standard IL-17 product (NIBSC code: 90/530) and the WHO standard IL-15 product (NIBSC code: 95/554), respectively.

In some embodiments, the biological activity in IU/mL is equivalent to (ED50 in ng/ml)1×10⁶. In particular embodiments, the ED50 (median effective dose that produces a quantal effect in 50% of a population to which it is administered) of recombinant human IL-2 or IL-15 is equivalent to the concentration required for the half-maximal stimulation of cell proliferation (XTT, or tetrazolium hydroxide, cleavage) with CTLL-2 (cytotoxic T cells derived from C57BL/6 mouse) cells. In certain embodiments, the ED50 of recombinant human IL-7 is equivalent to the concentration required for the half-maximal stimulation for proliferation of PHA (phytohaemagglutinin P)-activated human peripheral blood lymphocytes. Details relating to assays and calculations of IU for IL-2 are discussed in Wadhwa et al., Journal of Immunological Methods (2013), 379 (1-2): 1-7; and Gearing and Thorpe, Journal of Immunological Methods (1988), 114 (1-2): 3-9; details relating to assays and calculations of IU for IL-15 are discussed in Soman et al. Journal of Immunological Methods (2009) 348 (1-2): 83-94; hereby incorporated by reference in their entirety.

In some embodiments, the cells are incubated with a cytokine, e.g., a recombinant human cytokine, at a concentration of between 1 IU/ml and 1,000 IU/ml, between 10 IU/ml and 50 IU/ml, between 50 IU/ml and 100 IU/ml, between 100 IU/ml and 200 IU/ml, between 100 IU/ml and 500 IU/ml, between 250 IU/ml and 500 IU/ml, or between 500 IU/ml and 1,000 IU/ml.

In some embodiments, cells are incubated with IL-2 (e.g., human recombinant IL-2), at a concentration between 1 IU/ml and 200 IU/ml, between 10 IU/ml and 100 IU/ml, between 50 IU/ml and 150 IU/ml, between 80 IU/ml and 120 IU/ml, between 60 IU/ml and 90 IU/ml, or between 70 IU/ml and 90 IU/ml. In particular embodiments, the composition of enriched T cells is incubated with recombinant IL-2 at a concentration at or at about 50 IU/ml, 55 IU/ml, 60 IU/ml, 65 IU/ml, 70 IU/ml, 75 IU/ml, 80 IU/ml, 85 IU/ml, 90 IU/ml, 95 IU/ml, 100 IU/ml, 110 IU/ml, 120 IU/ml, 130 IU/ml, 140 IU/ml, or 150 IU/ml.

In some embodiments, cells are incubated with recombinant IL-7 (e.g., human recombinant IL-7), at a concentration between 100 IU/ml and 2,000 IU/ml, between 500 IU/ml and 1,000 IU/ml, between 100 IU/ml and 500 IU/ml, between 500 IU/ml and 750 IU/ml, between 750 IU/ml and 1,000 IU/ml, or between 550 IU/ml and 650 IU/ml. In particular embodiments, the cells are incubated with IL-7 at a concentration at or at about 50 IU/ml, 100 IU/ml, 150 IU/ml, 200 IU/ml, 250 IU/ml, 300 IU/ml, 350 IU/ml, 400 IU/ml, 450 IU/ml, 500 IU/ml, 550 IU/ml, 600 IU/ml, 650 IU/ml, 700 IU/ml, 750 IU/ml, 800 IU/ml, 750 IU/ml, 750 IU/ml, 750 IU/ml, or 1,000 IU/ml.

In some embodiments, cells are incubated with recombinant IL-15 (e.g., human recombinant IL-15), at a concentration between 0.1 IU/ml and 100 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 00 IU/ml. In particular embodiments, the cells are incubated with IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, or 50 IU/ml.

In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15.

In particular embodiments, the cells are incubated with the activation reagent in the presence of one or more antioxidants. In some embodiments, antioxidants include, but are not limited to, one or more antioxidants comprise a tocopherol, a tocotrienol, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), a flavonoids, an isoflavone, lycopene, beta-carotene, selenium, ubiquinone, luetin, S-adenosylmethionine, glutathione, taurine, N-acetyl cysteine (NAC), citric acid, L-carnitine, BHT, monothioglycerol, ascorbic acid, propyl gallate, methionine, cysteine, homocysteine, gluthatione, cystamine and cysstathionine, and/or glycine-glycine-histidine.

In some embodiments, the one or more antioxidants is or includes a sulfur containing oxidant. In certain embodiments, a sulfur containing antioxidant may include thiol-containing antioxidants and/or antioxidants which exhibit one or more sulfur moieties (e.g., within a ring structure). In some embodiments, the sulfur containing antioxidants may include, for example, N-acetylcysteine (NAC) and 2,3-dimercaptopropanol (DMP), L-2-oxo-4-thiazolidinecarboxylate (OTC) and lipoic acid. In particular embodiments, the sulfur containing antioxidant is a glutathione precursor. In some embodiments, the glutathione precursor is a molecule that may be modified in one or more steps within a cell to derived glutathione. In particular embodiments, a glutathione precursor may include, but is not limited to N-acetyl cysteine (NAC), L-2-oxothiazolidine-4-carboxylic acid (Procysteine), lipoic acid, S-allyl cysteine, or methylmethionine sulfonium chloride.

In some embodiments, incubating the cells under activation conditions includes incubating the cells in the presence of one or more antioxidants. In particular embodiments, the cells are stimulated with the activation reagent in the presence of one or more antioxidants. In some embodiments, the cells are incubated in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1 500 μg/ml and 2 mg/ml, 500 μg/ml and 5 mg/ml, between 1 mg/ml and 10 mg/ml, or between 1 mg/ml and 100 mg/ml of the one or more antioxidants. In some embodiments, the cells are incubated in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of the one or more antioxidant. In some embodiments, the one or more antioxidants is or includes a sulfur containing antioxidant. In particular embodiments, the one or more antioxidants is or includes a glutathione precursor.

In some embodiments, the one or more antioxidants is or includes N-acetyl cysteine (NAC). In some embodiments, incubating the cells under activation conditions includes incubating the cells in the presence of NAC. In particular embodiments, the cells are stimulated with the activation reagent in the presence of NAC. In some embodiments, the cells are incubated in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1 500 μg/ml and 2 mg/ml, 500 μg/ml and 5 mg/ml, between 1 mg/ml and 10 mg/ml, or between 1 mg/ml and 100 mg/ml of NAC. In some embodiments, the cells are incubated in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of NAC.

In some embodiments, the conditions for stimulation and/or activation can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the total duration of the incubation (e.g., with the activation agent), is between about 1 hour and 96 hours, 1 hour and 72 hours, 1 hour and 48 hours, 4 hours and 36 hours, 8 hours and 30 hours or 12 hours and 24 hours, such as at least 6 hours, 12 hours, 18 hours, 24 hours, 36 hours or 72 hours. In some embodiments, the further incubation is for a time between about 1 hour and 48 hours, 4 hours and 36 hours, 8 hours and 30 hours or 12 hours and 24 hours, inclusive.

In some embodiments, the cells are cultured, cultivated, and/or incubated under activation conditions prior to and/or during a step for introducing a polynucleotide, e.g., a polynucleotide encoding a recombinant receptor, to the cells, e.g., by transduction and/or transfection. In certain embodiments the cells are cultured, cultivated, and/or incubated under activation conditions for an amount of time between 30 minutes and 2 hours, between 1 hour and 8 hours, between 1 hour and 6 hours, between 6 hours and 12 hours, between 12 hours and 18 hours, between 16 hours and 24 hours, between 12 hours and 36 hours, between 24 hours and 48 hours, between 24 hours and 72 hours, between 42 hours and 54 hours, between 60 hours and 120 hours between 96 hours and 120 hours, between 90 hours and 110 hours, between 1 days and 7 days, between 3 days and 8 days, between 1 day and 3 days, between 4 days and 6 days, or between 4 days and 5 days prior to the transduction unit operation.

In certain embodiments, the cells are incubated with and/or in the presence of the activation reagent prior to and/or during the transduction unit operation the cells. In certain embodiments, the cells are incubated with and/or in the presence of the activation reagent for an amount of time between 12 hours and 36 hours, between 24 hours and 48 hours, between 24 hours and 72 hours, between 42 hours and 54 hours, between 60 hours and 120 hours between 96 hours and 120 hours, between 90 hours and between 2 days and 7 days, between 3 days and 8 days, between 1 day and 8 days, between 4 days and 6 days, or between 4 days and 5 days. In particular embodiments, the cells are cultured, cultivated, and/or incubated under activation conditions prior to and/or during the transduction unit operation the cells for an amount of time of less than 10 days, 9 days, 8 days, 7 days, 6 days, or 5 days, 4 days, or for an amount of time less than 168 hours, 162 hours, 156 hours, 144 hours, 138 hours, 132 hours, 120 hours, 114 hours, 108 hours, 102 hours, or 96 hours. In particular embodiments, the cells are incubated with and/or in the presence of the activation reagent for or for about 4 days, 5 days, 6 days, or 7 days.

In some embodiments, incubating the cells under activation conditions includes incubating the cells with an activation reagent. In some embodiments, the activation reagent contains or includes a bead, such as a paramagnetic bead, and the cells are incubated with the activation reagent at a ratio of less than 3:1 (beads:cells), such as a ratio of 1:1. In particular embodiments, the cells are incubated with the stimulatory/activation reagent in the presence of one or more cytokines and/or one or more antioxidants. In some embodiments, a composition of enriched CD4+ T cells is incubated with the activation reagent at a ratio of 1:1 (beads:cells) in the presence of recombinant IL-2, IL-7, IL-15, and NAC. In certain embodiments, a composition of enriched CD8+ T cells is incubated with the stimulatory reagent at a ratio of 1:1 (beads:cells) in the presence of recombinant IL-2, IL-15, and NAC. In some embodiments, the activation reagent is removed and/or separated from the cells at, within, or within about 6 days, 5 days, or 4 days from the start or initiation of the incubation (e.g., from the time the activation reagent is added to or contacted with the cells).

In some embodiments, incubating a composition of enriched cells under activation conditions is or includes incubating and/or contacting the composition of enriched cells with an activation reagent that is capable of activating and/or expanding T cells. In some embodiments, the activation reagent is capable of activation and/or activating one or more signals in the cells. In some embodiments, the one or more signals are mediated by a receptor. In particular embodiments, the one or more signals are, or are associated with, a change in signal transduction and/or a level or amount of secondary messengers, e.g., cAMP and/or intracellular calcium, a change in the amount, cellular localization, conformation, phosphorylation, ubiquitination, and/or truncation of one or more cellular proteins, and/or a change in a cellular activity, e.g., transcription, translation, protein degradation, cellular morphology, activation state, and/or cell division. In particular embodiments, the activation reagent activates and/or is capable of activating one or more intracellular signaling domains of one or more components of a T cell receptor (TCR) complex and/or one or more intracellular signaling domains of one or more costimulatory molecules.

In certain embodiments, the activation reagent contains a particle, e.g., a bead, that is conjugated or linked to one or more agents, e.g., biomolecules, that are capable of activating and/or expanding cells, e.g., T cells. In some embodiments, the one or more agents are bound to a bead. In some embodiments, the bead is biocompatible, i.e., composed of a material that is suitable for biological use. In some embodiments, the beads are non-toxic to cultured cells, e.g., cultured T cells. In some embodiments, the beads may be any particles which are capable of attaching agents in a manner that permits an interaction between the agent and a cell.

In some embodiments, an activation reagent contains one or more agents that are capable of activating and/or expanding cells (e.g., T cells), that are bound to or otherwise attached to a bead, for example to the surface of the bead. In certain embodiments, the bead is a non-cell particle. In particular embodiments, the bead may include a colloidal particle, a microsphere, nanoparticle, a magnetic bead, or the like. In some embodiments, the beads are agarose beads. In certain embodiments, the beads are sepharose beads.

In particular embodiments, the activation reagent contains beads that are monodisperse. In certain embodiments, beads that are monodisperse comprise size dispersions having a diameter standard deviation of less than 5% from each other.

In some embodiments, the bead contains one or more agent(s), such as an agent that is coupled, conjugated, or linked (directly or indirectly) to the surface of the bead. In some embodiments, an agent as contemplated herein can include, but is not limited to, RNA, DNA, proteins (e.g., enzymes), antigens, polyclonal antibodies, monoclonal antibodies, antibody fragments, carbohydrates, lipids lectins, or any other biomolecule with an affinity for a desired target. In some embodiments, the desired target is a T cell receptor and/or a component of a T cell receptor. In certain embodiments, the desired target is CD3. In certain embodiment, the desired target is a T cell costimulatory molecule, e.g., CD28, CD137 (4-1-BB), OX40, or ICOS. The one or more agents may be attached directly or indirectly to the bead by a variety of methods known and available in the art. The attachment may be covalent, noncovalent, electrostatic, or hydrophobic and may be accomplished by a variety of attachment means, including for example, a chemical means, a mechanical means, or an enzymatic means. In some embodiments, a biomolecule (e.g., a biotinylated anti-CD3 antibody) may be attached indirectly to the bead via another biomolecule (e.g., anti-biotin antibody) that is directly attached to the bead.

In some embodiments, the activation reagent contains a bead and one or more agents that directly interact with a macromolecule on the surface of a cell. In certain embodiments, the bead (e.g., a paramagnetic bead) interacts with a cell via one or more agents (e.g., an antibody) specific for one or more macromolecules on the cell (e.g., one or more cell surface proteins). In certain embodiments, the bead (e.g., a paramagnetic bead) is labeled with a first agent described herein, such as a primary antibody (e.g., an anti-biotin antibody) or other biomolecule, and then a second agent, such as a secondary antibody (e.g., a biotinylated anti-CD3 antibody) or other second biomolecule (e.g., streptavidin), is added, whereby the secondary antibody or other second biomolecule specifically binds to such primary antibodies or other biomolecule on the particle.

In some embodiments, the activation reagent contains one or more agent(s) (e.g. antibody) that is/are attached to a bead (e.g., a paramagnetic bead) and specifically binds to one or more of the following macromolecules on a cell (e.g., a T cell): CD2, CD3, CD4, CD5, CD8, CD25, CD27, CD28, CD29, CD31, CD44, CD45RA, CD45RO, CD54 (ICAM-1), CD127, MHCI, MHCII, CTLA-4, ICOS, PD-1, OX40, CD27L (CD70), 4-1BB (CD137), 4-1BBL, CD30L, LIGHT, IL-2R, IL-12R, IL-1R, IL-15R; IFN-gammaR, TNF-alphaR, IL-4R, IL-10R, CD18/CD11a (LFA-1), CD62L (L-selectin), CD29/CD49d (VLA-4), Notch ligand (e.g. Delta-like 1/4, Jagged 1/2, etc.), CCR1, CCR2, CCR3, CCR4, CCR5, CCR7, and CXCR3 or fragment thereof including the corresponding ligands to these macromolecules or fragments thereof. In some embodiments, an agent (e.g. antibody) attached to the bead specifically binds to one or more of the following macromolecules on a cell (e.g. a T cell): CD28, CD62L, CCR7, CD27, CD127, CD3, CD4, CD8, CD45RA, and/or CD45RO.

In some embodiments, one or more of the agents attached to the bead is an antibody. The antibody can include a polyclonal antibody, monoclonal antibody (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). In some embodiments, the activation reagent is an antibody fragment (including antigen-binding fragment), e.g., a Fab, Fab′-SH, Fv, scFv, or (Fab′)2 fragment. It will be appreciated that constant regions of any isotype can be used for the antibodies contemplated herein, including IgG, IgM, IgA, IgD, and IgE constant regions, and that such constant regions can be obtained from any human or animal species (e.g., murine species). In some embodiments, the agent is an antibody that binds to and/or recognizes one or more components of a T cell receptor. In particular embodiments, the agent is an anti-CD3 antibody. In certain embodiments, the agent is an antibody that binds to and/or recognizes a co-receptor. In some embodiments, the activation reagent comprises an anti-CD28 antibody.

In some embodiments, the bead has a diameter of greater than about 0.001 μm, greater than about 0.01 μm, greater than about 0.1 μm, greater than about 1.0 μm, greater than about 10 μm, greater than about 50 μm, greater than about 100 μm or greater than about 1000 μm and no more than about 1500 μm. In some embodiments, the bead has a diameter of about 1.0 μm to about 500 μm, about 1.0 μm to about 150 μm, about 1.0 μm to about 30 μm, about 1.0 μm to about 10 μm, about 1.0 μm to about 5.0 μm, about 2.0 μm to about 5.0 μm, or about 3.0 μm to about 5.0 μm. In some embodiments, the bead has a diameter of about 3 μm to about 5 μm. In some embodiments, the bead has a diameter of at least about 0.001 μm, 0.01 μm, 0.1 μm, 0.5 μm, 1.0 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 12 μm, 14 μm, 16 μm, 18 μm or 20 μm. In certain embodiments, the bead has a diameter of, or about, 4.5 μm. In certain embodiments, the bead has a diameter of, or about, 2.8 μm.

In some embodiments, the beads have a density of greater than 0.001 g/cm³, greater than 0.01 g/cm³, greater than 0.05 g/cm³, greater than 0.1 g/cm³, greater than 0.5 g/cm³, greater than 0.6 g/cm³, greater than 0.7 g/cm³, greater than 0.8 g/cm³, greater than 0.9 g/cm³, greater than 1 g/cm³, greater than 1.1 g/cm³, greater than 1.2 g/cm³, greater than 1.3 g/cm³, greater than 1.4 g/cm³, greater than 1.5 g/cm³, greater than 2 g/cm³, greater than 3 g/cm³, greater than 4 g/cm³, or greater than 5 g/cm³. In some embodiments, the beads have a density of between about 0.001 g/cm³ and about 100 g/cm³, about 0.01 g/cm³ and about 50 g/cm³, about 0.1 g/cm³ and about 10 g/cm³, about 0.1 g/cm³ and about 0.5 g/cm³, about 0.5 g/cm³ and about 1 g/cm³, about 0.5 g/cm³ and about 1.5 g/cm³, about 1 g/cm³ and about 1.5 g/cm³, about 1 g/cm³ and about 2 g/cm³, or about 1 g/cm³ and about 5 g/cm³. In some embodiments, the beads have a density of about 0.5 g/cm³, about 0.5 g/cm³, about 0.6 g/cm³, about 0.7 g/cm³, about 0.8 g/cm³, about 0.9 g/cm³, about 1.0 g/cm³, about 1.1 g/cm³, about 1.2 g/cm³, about 1.3 g/cm³, about 1.4 g/cm³, about 1.5 g/cm³, about 1.6 g/cm³, about 1.7 g/cm³, about 1.8 g/cm³, about 1.9 g/cm³, or about 2.0 g/cm³. In certain embodiments, the beads have a density of about 1.6 g/cm³. In particular embodiments, the beads or particles have a density of about 1.5 g/cm3. In certain embodiments, the particles have a density of about 1.3 g/cm³. In certain embodiments, a plurality of the beads has a uniform density. In certain embodiments, a uniform density comprises a density standard deviation of less than 10%, less than 5%, or less than 1% of the mean bead density. In some embodiments, the beads have a surface area of between about 0.001 m² per each gram of particles (m²/g) to about 1,000 m²/g, about 0.010 m²/g to about 100 m²/g, about 0.1 m²/g to about 10 m²/g, about 0.1 m²/g to about 1 m²/g, about 1 m²/g to about 10 m²/g, about 10 m²/g to about 100 m²/g, about 0.5 m²/g to about 20 m²/g, about 0.5 m²/g to about 5 m²/g, or about 1 m²/g to about 4 m²/g. In some embodiments, the particles or beads have a surface area of about 1 m²/g to about 4 m²/g.

In some embodiments, the bead contains at least one material at or near the bead surface that can be coupled, linked, or conjugated to an agent. In some embodiments, the bead is surface functionalized, i.e. comprises functional groups that are capable of forming a covalent bond with a binding molecule, e.g., a polynucleotide or a polypeptide. In particular embodiments, the bead comprises surface-exposed carboxyl, amino, hydroxyl, tosyl, epoxy, and/or chloromethyl groups. In particular embodiments, the beads comprise surface exposed agarose and/or sepharose. In certain embodiments, the bead surface comprises attached activation reagents that can bind or attach binding molecules. In particular embodiments, the biomolecules are polypeptides. In some embodiments, the beads comprise surface exposed protein A, protein G, or biotin.

In some embodiments, the bead reacts or is responsive in or to a magnetic field. In some embodiments, the bead is a magnetic bead. In some embodiments, the magnetic bead is paramagnetic. In particular embodiments, the magnetic bead is superparamagnetic. In certain embodiments, the beads do not display any magnetic properties unless they are exposed to a magnetic field.

In particular embodiments, the bead comprises a magnetic core, a paramagnetic core, or a superparamagnetic core. In some embodiments, the magnetic core contains a metal. In some embodiments, the metal can be, but is not limited to, iron, nickel, copper, cobalt, gadolinium, manganese, tantalum, zinc, zirconium or any combinations thereof. In certain embodiments, the magnetic core comprises metal oxides (e.g., iron oxides), ferrites (e.g., manganese ferrites, cobalt ferrites, nickel ferrites, etc.), hematite and metal alloys (e.g., CoTaZn). In some embodiments, the magnetic core comprises one or more of a ferrite, a metal, a metal alloy, an iron oxide, or chromium dioxide. In some embodiments, the magnetic core comprises elemental iron or a compound thereof. In some embodiments, the magnetic core comprises one or more of magnetite (Fe₃O₄), maghemite (γFe₂O₃), or greigite (Fe₃S₄). In some embodiments, the inner core comprises an iron oxide (e.g., Fe₃O₄).

In certain embodiments, the bead contains a magnetic, paramagnetic, and/or superparamagnetic core that is covered by a surface functionalized coat or coating. In some embodiments, the coat can contain a material that can include, but is not limited to, a polymer, a polysaccharide, a silica, a fatty acid, a protein, a carbon, agarose, sepharose, or a combination thereof. In some embodiments, the polymer can be a polyethylene glycol, poly (lactic-co-glycolic acid), polyglutaraldehyde, polyurethane, polystyrene, or a polyvinyl alcohol. In certain embodiments, the outer coat or coating comprises polystyrene. In particular embodiments, the outer coating is surface functionalized.

In some embodiments, the activation reagent comprises a bead that contains a metal oxide core (e.g., an iron oxide core) and a coat, wherein the metal oxide core comprises at least one polysaccharide (e.g., dextran), and wherein the coat comprises at least one polysaccharide (e.g., amino dextran), at least one polymer (e.g., polyurethane) and silica. In some embodiments, the metal oxide core is a colloidal iron oxide core. In certain embodiments, the one or more agents include an antibody or antigen-binding fragment thereof. In particular embodiments, the one or more agents include an anti-CD3 antibody and an anti-CD28 antibody. In some embodiments, the activation reagent comprises an anti-CD3 antibody, anti-CD28 antibody, and an anti-biotin antibody. In some embodiments, the activation reagent comprises an anti-biotin antibody. In some embodiments, the bead has a diameter of about 3 μm to about 10 μm. In some embodiments, the bead has a diameter of about 3 μm to about 5 μm. In certain embodiments, the bead has a diameter of about 3.5 μm.

In some embodiments, the activation reagent comprises one or more agents that are attached to a bead comprising a metal oxide core (e.g., an iron oxide inner core) and a coat (e.g., a protective coat), wherein the coat comprises polystyrene. In certain embodiments, the beads are monodisperse, paramagnetic (e.g., superparamagnetic) beads comprising a paramagnetic (e.g., superparamagnetic) iron core, e.g., a core comprising magnetite (Fe₃O₄) and/or maghemite (γFe₂O₃) and a polystyrene coat or coating. In some embodiments, the bead is non-porous. In some embodiments, the beads contain a functionalized surface to which the one or more agents are attached. In certain embodiments, the one or more agents are covalently bound to the beads at the surface. In some embodiments, the one or more agents include an antibody or antigen-binding fragment thereof. In some embodiments, the one or more agents include an anti-CD3 antibody and an anti-CD28 antibody. In some embodiments, the one or more agents include an anti-CD3 antibody and/or an anti-CD28 antibody, and an antibody or antigen fragment thereof capable of binding to a labeled antibody (e.g., biotinylated antibody), such as a labeled anti-CD3 or anti-CD28 antibody. In certain embodiments, the beads have a density of about 1.5 g/cm³ and a surface area of about 1 m²/g to about 4 m²/g. In particular embodiments; the beads are monodisperse superparamagnetic beads that have a diameter of about 4.5 μm and a density of about 1.5 g/cm³. In some embodiments, the beads the beads are monodisperse superparamagnetic beads that have a mean diameter of about 2.8 μm and a density of about 1.3 g/cm³.

In some embodiments, the cells are incubated with activation reagent a ratio of beads to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of activation reagent to cells is about 1:1 or is 1:1.

In particular embodiments, the stimulatory reagent contains an oligomeric reagent (e.g., a streptavidin mutein reagent), that is conjugated, linked, or attached to one or more agent(s) (e.g., ligand), which is capable of activating an intracellular signaling domain of a TCR complex. In some embodiments, the one or more agents have an attached binding domain or binding partner (e.g., a binding partner C) that is capable of binding to oligomeric reagent at a particular binding sites (e.g., binding site Z). In some embodiments, a plurality of the agent is reversibly bound to the oligomeric reagent. In various embodiments, the oligomeric reagent has a plurality of the particular binding sites which, in certain embodiments, are reversibly bound to a plurality of agents at the binding domain (e.g., binding partner C). In some embodiments, the amount of bound agents are reduced or decreased in the presence of a competition reagent, e.g., a reagent that is also capable of binding to the particular binding sites (e.g., binding site Z).

In some embodiments, the stimulatory reagent is or includes a reversible system in which at least one agent (e.g., an agent that is capable of producing a signal in a cell such as a T cell) is associated (e.g., reversibly associated), with the oligomeric reagent. In some embodiments, the reagent contains a plurality of binding sites capable of binding (e.g., reversibly binding), to the agent. In some cases, the reagent is an oligomeric particle reagent having at least one attached agent capable of producing a signal in a cell such as a T cell. In some embodiments, the agent contains at least one binding site (e.g., a binding site B), that can specifically bind an epitope or region of the molecule and also contains a binding partner, also referred to herein as a binding partner C, that specifically binds to at least one binding site of the reagent (e.g., binding site Z) of the reagent. In some embodiments, the binding interaction between the binding partner C and the at least one binding site Z is a non-covalent interaction. In some cases, the binding interaction between the binding partner C and the at least one binding site Z is a covalent interaction. In some embodiments, the binding interaction, such as non-covalent interaction, between the binding partner C and the at least one binding site Z is reversible.

Substances that may be used as oligomeric reagents in such reversible systems are known, see e.g., U.S. Pat. Nos. 5,168,049; 5,506,121; 6,103,493; 7,776,562; 7,981,632; 8,298,782; 8,735,540; 9,023,604; and International published PCT Appl. Nos. WO2013/124474 and WO2014/076277. Non-limiting examples of reagents and binding partners capable of forming a reversible interaction, as well as substances (e.g. competition reagents) capable of reversing such binding, are described below.

In some embodiments, the oligomeric reagent is an oligomer of streptavidin, streptavidin mutein or analog, avidin, an avidin mutein or analog (such as neutravidin) or a mixture thereof, in which such oligomeric reagent contains one or more binding sites for reversible association with the binding domain of the agent (e.g., a binding partner C). In some embodiments, the binding domain of the agent can be a biotin, a biotin derivative or analog, or a streptavidin-binding peptide or other molecule that is able to specifically bind to streptavidin, a streptavidin mutein or analog, avidin or an avidin mutein or analog.

In certain embodiments, one or more agents (e.g., agents that are capable of producing a signal in a cell such as a T cell) associate with, such as are reversibly bound to, the oligomeric reagent, such as via the plurality of the particular binding sites (e.g., binding sites Z) present on the oligomeric reagent. In some cases, this results in the agents being closely arranged to each other such that an avidity effect can take place if a target cell having (at least two copies of) a cell surface molecule that is bound by or recognized by the agent is brought into contact with the agent.

In some embodiments, the oligomeric reagent is a streptavidin oligomer, a streptavidin mutein oligomer, a streptavidin analog oligomer, an avidin oligomer, an oligomer composed of avidin mutein or avidin analog (such as neutravidin) or a mixture thereof. In particular embodiments, the oligomeric reagents contain particular binding sites that are capable of binding to a binding domain (e.g., the binding partner C) of an agent. In some embodiments, the binding domain can be a biotin, a biotin derivative or analog, or a streptavidin-binding peptide or other molecule that is able to specifically bind to streptavidin, a streptavidin mutein or analog, avidin or an avidin mutein or analog.

In some embodiments, the streptavidin can be wild-type streptavidin, streptavidin muteins or analogs, such as streptavidin-like polypeptides. Likewise, avidin, in some aspects, includes wild-type avidin or muteins or analogs of avidin such as neutravidin, a deglycosylated avidin with modified arginines that typically exhibits a more neutral isoelectric point (pI) and is available as an alternative to native avidin. Generally, deglycosylated, neutral forms of avidin include those commercially available forms such as “Extravidin”, available through Sigma Aldrich (St. Louis, Mo.), or “NeutrAvidin” available from Thermo Scientific (Waltham, Mass.) or Invitrogen (Carlsbad, Calif.), for example

In some embodiments, the reagent is a streptavidin or a streptavidin mutein or analog. In some embodiments, wild-type streptavidin (wt-streptavidin) has the amino acid sequence disclosed by Argarana et al, Nucleic Acids Res. 14 (1986) 1871-1882 (SEQ ID NO: 1). In general, streptavidin naturally occurs as a tetramer of four identical subunits, i.e. it is a homo-tetramer, where each subunit contains a single binding site for biotin, a biotin derivative or analog or a biotin mimic. An exemplary sequence of a streptavidin subunit is the sequence of amino acids set forth in SEQ ID NO: 1, but such a sequence also can include a sequence present in homologs thereof from other Streptomyces species. In particular, each subunit of streptavidin may exhibit a strong binding affinity for biotin with a dissociation constant (Kd) on the order of about 10⁻¹⁴M. In some cases, streptavidin can exist as a monovalent tetramer in which only one of the four binding sites is functional (Howarth et al. (2006) Nat. Methods, 3:267-73; Zhang et al. (2015) Biochem. Biophys. Res. Commun., 463:1059-63)), a divalent tetramer in which two of the four binding sites are functional (Fairhead et al. (2013) J. Mol. Biol., 426:199-214), or can be present in monomeric or dimeric form (Wu et al. (2005) J. Biol. Chem., 280:23225-31; Lim et al. (2010) Biochemistry, 50:8682-91).

In some embodiments, streptavidin may be in any form, such as wild-type or unmodified streptavidin, such as a streptavidin from a Streptomyces species or a functionally active fragment thereof that includes at least one functional subunit containing a binding site for biotin, a biotin derivative or analog or a biotin mimic, such as generally contains at least one functional subunit of a wild-type streptavidin from Streptomyces avidinii set forth in SEQ ID NO: 1 or a functionally active fragment thereof. For example, in some embodiments, streptavidin can include a fragment of wild-type streptavidin, which is shortened at the N- and/or C-terminus. Such minimal streptavidins include any that begin N-terminally in the region of amino acid positions 10 to 16 of SEQ ID NO: 1 and terminate C-terminally in the region of amino acid positions 133 to 142 of SEQ ID NO: 1. In some embodiments, a functionally active fragment of streptavidin contains the sequence of amino acids set forth in SEQ ID NO: 2. In some embodiments, streptavidin, such as set forth in SEQ ID NO: 2, can further contain an N-terminal methionine at a position corresponding to Ala13 with numbering set forth in SEQ ID NO: 1. Reference to the position of residues in streptavidin or streptavidin muteins is with reference to numbering of residues in SEQ ID NO: 1.

Examples of streptavidins or streptavidin muteins are mentioned, for example, in WO 86/02077, DE 19641876 A1, U.S. Pat. No. 6,022,951, WO 98/40396 or WO 96/24606. Examples of streptavidin muteins are known in the art, see e.g., U.S. Pat. Nos. 5,168,049; 5,506,121; 6,022,951; 6,156,493; 6,165,750; 6,103,493; or 6,368,813; or International published PCT App. No. WO2014/076277.

In some embodiments, a streptavidin mutein can contain amino acids that are not part of an unmodified or wild-type streptavidin or can include only a part of a wild-type or unmodified streptavidin. In some embodiments, a streptavidin mutein contains at least one subunit that can have one more amino acid substitutions (replacements) compared to a subunit of an unmodified or wild-type streptavidin, such as compared to the wild-type streptavidin subunit set forth in SEQ ID NO: 1 or a functionally active fragment thereof, e.g. set forth in SEQ ID NO: 2.

In some embodiments, the binding affinity, such as dissociation constant (K_{d}), of streptavidin or a streptavidin mutein for a binding domain is less than 1×10⁻⁴M, 5×10⁻⁴ M, 1×10⁻⁵M, 5×10⁻⁵M, 1×10⁻⁶ M, 5×10⁻⁶ M or 1×10⁻⁷ M, but generally greater than 1×10⁻¹³ M, 1×10⁻¹² M or 1×10⁻¹¹M. For example, peptide sequences (Strep-tags), such as disclosed in U.S. Pat. No. 5,506,121, can act as biotin mimics and demonstrate a binding affinity for streptavidin (e.g., with a K_{d} of approximately between 10⁻⁴ and 10⁻⁵M). In some cases, the binding affinity can be further improved by making a mutation within the streptavidin molecule, see e.g. U.S. Pat. No. 6,103,493 or International published PCT App. No. WO2014/076277. In some embodiments, binding affinity can be determined by methods known in the art, such as any described herein.

In some embodiments, the reagent, such as a streptavidin or streptavidin mutein, exhibits binding affinity for a peptide ligand binding partner, which peptide ligand binding partner can be the binding partner C present in the agent (e.g., receptor-binding agent or selection agent). In some embodiments, the peptide sequence contains a sequence with the general formula His-Pro-Xaa, where Xaa is glutamine, asparagine, or methionine, such as contains the sequence set forth in SEQ ID NO: 3. In some embodiments, the peptide sequence has the general formula set forth in SEQ ID NO: 4, or the general formula such as set forth in SEQ ID NO: 5. In one example, the peptide sequence is Trp-Arg-His-Pro-Gln-Phe-Gly-Gly (also called Strep-Tag®, set forth in SEQ ID NO: 6). In one example, the peptide sequence is Trp-Ser-His-Pro-Gln-Phe-Glu-Lys (also called Strep-Tag® II, set forth in SEQ ID NO: 7). In some embodiments, the peptide ligand contains a sequential arrangement of at least two streptavidin-binding modules, wherein the distance between the two modules is at least 0 and not greater than 50 amino acids, wherein one binding module has 3 to 8 amino acids and contains at least the sequence His-Pro-Xaa, where Xaa is glutamine, asparagine, or methionine, and wherein the other binding module has the same or different streptavidin peptide ligand, such as set forth in SEQ ID NO: 4 (see e.g. International Published PCT Appl. No. WO02/077018; U.S. Pat. No. 7,981,632). In some embodiments, the peptide ligand contains a sequence having the formula set forth in any of SEQ ID NO: 8 or 9. In some embodiments, the peptide ligand has the sequence of amino acids set forth in any of SEQ ID NOS: 10-12, 13-14. In most cases, all these streptavidin binding peptides bind to the same binding site, namely the biotin binding site of streptavidin. If one or more of such streptavidin binding peptides is used as binding partners C, e.g. C1 and C2, the multimerization reagent and/or oligomeric particle reagents bound to the one or more agents via the binding partner C is typically composed of one or more streptavidin muteins.

In some embodiments, the streptavidin mutein is a mutant as described in U.S. Pat. No. 6,103,493. In some embodiments, the streptavidin mutein contains at least one mutation within the region of amino acid positions 44 to 53, based on the amino acid sequence of wild-type streptavidin, such as set forth in SEQ ID NO: 1. In some embodiments, the streptavidin mutein contains a mutation at one or more residues 44, 45, 46, and/or 47. In some embodiments, the streptavidin mutein contains a replacement of Glu at position 44 of wild-type streptavidin with a hydrophobic aliphatic amino acid (e.g. Val, Ala, Ile or Leu), any amino acid at position 45, an aliphatic amino acid, such as a hydrophobic aliphatic amino acid at position 46 and/or a replacement of Val at position 47 with a basic amino acid, e.g. Arg or Lys, such as generally Arg. In some embodiments, Ala is at position 46 and/or Arg is at position 47 and/or Val or Ile is at position 44. In some embodiments, the streptavidin mutant contains residues Val44-Thr45-Ala46-Arg47, such as set forth in exemplary streptavidin muteins containing the sequence of amino acids set forth in SEQ ID NO: 15 or SEQ ID NO: 16 or 17 (also known as streptavidin mutant 1, SAM1). In some embodiments, the streptavidin mutein contains residues Ile44-Gly45-Ala46-Arg47, such as set forth in exemplary streptavidin muteins containing the sequence of amino acids set forth in SEQ ID NO: 18, 19, or 20 (also known as SAM2). In some cases, such streptavidin mutein are described, for example, in U.S. Pat. No. 6,103,493, and are commercially available under the trademark Strep-Tactin®. In some embodiments, the mutein streptavidin contains the sequence of amino acids set forth in SEQ ID NO: 21 or SEQ ID NO: 22. In particular embodiments, the molecule is a tetramer of streptavidin or a streptavidin mutein comprising a sequence set forth in any of SEQ ID NOS: 2, 16, 19, 21, 23, 17 or 20, which, as a tetramer, is a molecule that contains 20 primary amines, including 1 N-terminal amine and 4 lysines per monomer.

In some embodiments, streptavidin mutein exhibits a binding affinity characterized by a dissociation constant (K_{d}) that is or is less than 3.7×10⁻⁵M for the peptide ligand (Trp-Arg-His-Pro-Gln-Phe-Gly-Gly; also called Strep-Tag®, set forth in SEQ ID NO: 6) and/or that is or is less than 7.1×10⁻⁵M for the peptide ligand (Trp-Ser-His-Pro-Gln-Phe-Glu-Lys; also called Strep-Tag® II, set forth in SEQ ID NO: 7) and/or that is or is less than 7.0×10⁻⁵M, 5.0×10⁻⁵M, 1.0×10⁻⁵ M, 5.0×10⁻⁶ M, 1.0×10⁻⁶ M, 5.0×10⁻⁷ M, or 1.0×10⁻⁷ M, but generally greater than 1×10⁻¹³ M, 1×10⁻¹²M or 1×10⁻¹¹M for any of the peptide ligands set forth in any of SEQ ID NOS: 7, 8, 9, 13, 14, 10-12, 5, 6, 3, 4.

In some embodiments, the resulting streptavidin mutein exhibits a binding affinity characterized by an association constant (Kₐ) that is or is greater than 2.7×10⁴M⁻¹ for the peptide ligand (Trp-Arg-His-Pro-Gln-Phe-Gly-Gly; also called Strep-Tag®, set forth in SEQ ID NO: 6) and/or that is or is greater than 1.4×10⁴ M⁻¹ for the peptide ligand (Trp-Ser-His-Pro-Gln-Phe-Glu-Lys; also called Strep-Tag® II, set forth in SEQ ID NO: 7) and/or that is or is greater than 1.43×10⁴M⁻¹, 1.67×10⁴M⁻¹, 2×10⁴M⁻¹, 3.33×10⁴M⁻¹, 5×10⁴ M⁻¹, 1×10⁵ M⁻¹, 1.11×10⁵M⁻¹, 1.25×10⁵M⁻¹, 1.43×10⁵M⁻¹, 1.67×10⁵M⁻¹, 2×10⁵M⁻¹, 3.33×10⁵M⁻¹, 5×10⁵ M⁻¹, 1×10⁶M⁻¹, 1.11×10⁶M⁻¹, 1.25×10⁶M⁻¹, 1.43×10⁶M⁻¹, 1.67×10⁶M⁻¹, 2×10⁶M⁻¹, 3.33×10⁶M⁻¹, 5×10⁶M⁻¹, 1×10⁷M⁻¹, but generally less than 1×10¹³ M⁻¹, 1×10¹² M⁻¹ or 1×10¹¹M⁻¹ for any of the peptide ligands set forth in any of SEQ ID NOS: 7, 8, 9, 13, 14, 10-12, 5, 6, 3, 4.

In particular embodiments, provided herein is an oligomeric particle reagent that is composed of and/or contains a plurality of streptavidin or streptavidin mutein tetramers. In certain embodiments, the oligomeric particle reagent provided herein contains a plurality of binding sites that reversibly bind or are capable of reversibly binding to one or more agents, e.g., a stimulatory agent and/or a selection agent. In some embodiments, the oligomeric particle has a radius (e.g., an average radius), of between 70 nm and 125 nm, inclusive; a molecular weight of between 1×10⁷ g/mol and 1×10⁹ g/mol, inclusive; and/or between 1,000 and 5,000 streptavidin or streptavidin mutein tetramers, inclusive. In some embodiments, the oligomeric particle reagent is bound (e.g., reversibly bound), to one or more agents such as an agent that binds to a molecule (e.g. receptor), on the surface of a cell. In certain embodiments, the one or more agents are agents described herein. In some embodiments, the agent is an anti-CD3 and/or an anti-CD28 antibody or antigen binding fragment thereof, such as an antibody or antigen fragment thereof that contains a binding partner, e.g., a streptavidin binding peptide, e.g. Strep-Tag® II. In particular embodiments, the one or more agents is an anti-CD3 and/or an anti CD28 Fab containing a binding partner, e.g., a streptavidin binding peptide, e.g. Strep-Tag® II.

In some embodiments, provided herein is an oligomeric particle reagent that is composed of and/or contains a plurality of streptavidin or streptavidin mutein tetramers. In certain embodiments, the oligomeric particle reagent provided herein contains a plurality of binding sites that reversibly bind or are capable of reversibly binding to one or more agents (e.g., a stimulatory agent and/or a selection agent). In some embodiments, the oligomeric particle has a radius (e.g., an average radius), of between 80 nm and 120 nm, inclusive; a molecular weight (e.g., an average molecular weight) of between 7.5×10⁶ g/mol and 2×10⁸ g/mol, inclusive; and/or an amount (e.g., an average amount), of between 500 and 10,000 streptavidin or streptavidin mutein tetramers, inclusive. In some embodiments, the oligomeric particle reagent is bound (e.g., reversibly bound), to one or more agents, such as an agent that binds to a molecule, e.g. receptor, on the surface of a cell. In certain embodiments, the one or more agents are agents described herein. In some embodiments, the agent is an anti-CD3 and/or an anti-CD28 Fab, such as a Fab that contains a binding partner (e.g., a streptavidin binding peptide, for example Strep-Tag® II). In particular embodiments, the one or more agents is an anti-CD3 and/or an anti CD28 Fab containing a binding partner (e.g., a streptavidin binding peptide, for example Strep-Tag® II).

In some embodiments, the cells are stimulated in the presence of, of about, or of at least 0.01 μg, 0.02 μg, 0.03 μg, 0.04 μg, 0.05 μg, 0.1 μg, 0.2 μg, 0.3 μg, 0.4 μg, 0.5 μg, 0.75 μg, 1 μg, 2 μg, 3 μg, 4 μg, 5 μg, 6 μg, 7 μg, 8 μg, 9 μg, or 10 μg of the oligomeric stimulatory reagent per 10⁶ cells. In some embodiments, the cells are stimulated in the presence of or of about 4 μg per 10⁶ cells. In particular embodiments, the cells are stimulated in the presence of or of about 0.8 μg per 10⁶ cells. In certain aspects, 4 μg of the oligomeric stimulatory reagent is or includes 3 μg of oligomeric particles and 1 μg of attached agents (e.g., 0.5 μg of anti-CD3 Fabs and 0.5 μg of anti-CD28 Fabs).

Transduction/Transfection Unit Operation

The transduction/transfection method set forth below is configured to run a system with 48 total conditions when cells are activated with a 6-well plate. However, this can be expanded or contracted with different systems and/or system components, for example T cell activation for up to 192 conditions to be processed in parallel. This unit operation allows the user to input a forward processing or target total nucleated cell (TNC) processing of activated material preference. The user also inputs the number of transduction well replicates. For reps=1, one 24-well centrifuge plate is required for every 4× plates. For reps=2, 1×24-well centrifuge plate is required for 1× plate. Here, the desired amount is transferred to a 24-well flat bottom plate or a 24-well pyramid or round-bottom deep well plate for spinoculation. Post transduction, the cells can either be incubated in a 24-well plate for next day inoculation or transferred to a 24-deepwell expansion plate for inoculation, according to the user's preference. The discussion here centers on a viral transduction approach, however it is contemplated, as will be elaborated in greater detail below, that other methods for introducing recombinant DNA into activated T cells are encompassed by the present disclosure. For example, one or more of electroporation, reagent-based transfection, cell compression, or squeezing can be relied upon for incorporating the recombinant DNA into the activated T cells, without departing from the scope of this disclosure.

With reference to FIG. 17, once the activation unit operation 210 is concluded, the control system initiates the transduction/transfection unit operation 220. The transduction unit operation 220 begins with a worktable set up. In embodiments, a user is prompted to setup worktable 60 with DiTi, reagent troughs, cell culture media, cell counting reagent, etc. In embodiments, a user is prompted to place balanced 24-well flat bottom and 24-deepwell plates onto the worktable 60. 24-well flat bottom plates are used for T cell spinoculation. 24-deepwell plates are used for cell centrifugation. In embodiments, a user is prompted to input transduction mode—set transduction TNC or forward processing. In embodiments, a user is prompted to input post transduction mode—inoculation or incubation post transduction. In embodiments, a user is prompted to input condition number, viral vector volume, number of transduction replicates, spinoculation volume, incubation/inoculation volume. In embodiments, a user is prompted to input total sampling and analytical sample (e.g., amino acid analysis (AAA)/flow cytometry) volumes. The user can be prompted to enter these parameters in real time, or as part of a script, for example, set up by the user prior to initiation of the system 10 of the method 200 as shown in FIG. 17. In embodiments, a user is prompted to setup worktable 60 with “n” number of 24-well flat bottom plates based on the number of conditions input. In embodiments, a user is prompted to setup worktable 60 with sampling and “n” number of plates based on the number of conditions input. Sampling plates include a 96-deepwell plate, a 96-well low attachment plate (cell counting), and a 96-well round bottom plate (AAA/flow cytometry).

Once the worktable 60 setup is complete, sampling is initiated by the control system 20. Plates are unlidded, each sample well is mixed, and a total sampling volume is aspirated per sample and dispensed into the 96-deepwell plate. Plates are relidded. The dispensed sampling volume is then mixed, and aliquoted into the cell counting and AAA/flow cytometry plates. The cell counting reagent is then dispensed into the low attachment cell counting plates according the number of conditions input. In certain embodiments, the cell counts for the sampling plates are automatically read by the cell counting module 75. In other embodiments, the sampling plates are then brought to the front of the worktable 60 for user reachability, then removed from the worktable 60 by a user for manual cell counting. Cell concentration measurements are obtained by the system controller 20, either automatically from the cell counting module 75 or as manually entered by a user.

Once the sampling is complete, preparation for spinoculation is initiated by the control system 20. The user is prompted to place viral vector into a 25 mL trough. According to user input, the user is then optionally prompted to place “n” number of 24-deepwell centrifuge plates on the worktable 60 according the number of conditions input. The container manipulation module 50 unlids both the centrifuge plates and the plates, then aspirates either the required cell number to reach the transduction TNC or the entire sample and dispenses it into the centrifuge plates. The container manipulation module 50 lids the centrifuge plates with eccentric fingers 52, then using a finger exchange system (FES), replaces the fingers with centric fingers 54. The container manipulation module 50, such as an RGA, then transports the centrifuge plates and its balance plate (only with odd number of 24-deepwell centrifuge plates) vertically into the robotic centrifuge 65. Post centrifugation, the centrifuge plates are returned to the worktable 60. The container manipulation module 50, such as an RGA, unlids the centrifuge plates, followed by flexible liquid manipulation module 40, such as FCA, aspiration of the supernatants per condition well. Aspiration is performed at slower speed with a well offset to ensure that the cell pellet is not disturbed. Supernatant aspiration proceeds until the spinoculation volume input is left in each well. In some embodiments, the spinoculation step is performed in the original vessel and there may not be a cell transfer step. In this case, a volume reduction occurs in the original vessel to obtain the desired cell concentration before proceeding.

Once preparation for spinoculation is complete, the spinoculation module is initiated by the control system 20. The container manipulation module 50, such as an RGA, with eccentric fingers 52, obtains 24-well flat bottom plates from hotels 105 and places them on the worktable 60 nests according the number of plates required for the condition number input. The flexible liquid manipulation module 40 proceeds with mixing each well of the 24-deepwell centrifuge plates. It then aspirates the well contents and dispenses into a 24-well flat bottom plates. Once the predetermined number of cells have been dispensed into the 24-well flat bottom spinoculation plates, viral vector is dispensed per well according to the volume input. The container manipulation module 50, such as an RGA, with eccentric fingers 52, lids the 24-well flat bottom plates, then using FES, replaces the eccentric fingers with centric fingers. The container manipulation module 50, then transports the 24-well flat bottom plates and its balance plate (only with odd number of 24-well flat bottom plates) vertically into the robotic centrifuge 65 for spinoculation.

Once the spinoculation is complete, cell incubation post transduction is initiated by the control system 20, if transduction is followed by incubation according to user input. Post centrifugation, the 24-well flat bottom plates are returned to the worktable 60. The container manipulation module 50, unlids the 24-well flat bottom plates, then the flexible liquid manipulation module 40, such as a FCA, dispenses fresh media to each condition well to reach the incubation volume. The flexible liquid manipulation module 40, such as a FCA, then mixes the plate wells according to the number of conditions. The container manipulation module 50, such as an RGA, using eccentric fingers 52, lids all 24-well flat bottom plates on the worktable 60. In embodiments, the plates are automatically transferred to a mammalian cell incubator for inoculation. In embodiments, the all remaining labware is automatically removed from the worktable. In embodiments, a user is prompted to place plates into the incubator for inoculation. In embodiments a user is prompted remove all remaining labware from the worktable. The user is then prompted to remove all remaining labware from the worktable 60.

If cell inoculation post transduction is chosen according to user input, post centrifugation, the 24-well flat bottom plates are returned to the worktable 60. The user is then prompted to place 24-deepwell expansion plates onto the worktable 60. The container manipulation module 50, such as an RGA, using eccentric fingers 52, unlids the expansion plates. The flexible liquid manipulation module 40 follows by dispensing balance cell culture media into each well of the 24-deepwell expansion plate per condition according the volume input. The flexible liquid manipulation module 40, such as FCA returns to the 24-well flat bottom plate, mixes the plate wells according to the number of conditions, then aspirates the cell culture contents per well and dispenses it into the 24-deepwell expansion plates. The container manipulation module 50, using eccentric fingers 52, lids all 24-deepwell expansion plates on the worktable 60. In embodiments, the plates are automatically transferred to a mammalian cell incubator for inoculation. In embodiments, the all remaining labware is automatically removed from the worktable. In embodiments, a user is prompted to place plates into the incubator for inoculation. In embodiments, a user is prompted remove all remaining labware from the worktable 60.

In embodiments, the methods provided herein are used for the transduction or transfection of a polynucleotide, e.g., a recombinant polynucleotide encoding a recombinant protein. In particular embodiments, the recombinant proteins are recombinant receptors.

Various methods for the introduction of polynucleotide (e.g., recombinant polynucleotides) encoding one or more recombinant proteins (e.g., CARs or TCRs), are known and may be used with the provided systems, methods and compositions. Exemplary methods include those for transfer of nucleic acids encoding the polypeptides or receptors, including via viral vectors, e.g., retroviral or lentiviral, non-viral vectors or transposons, e.g., Sleeping Beauty transposon system. Methods of gene transfer can include transduction, electroporation or other methods that result into gene transfer into the cell, or any delivery methods described herein. Other approaches and vectors for transfer of the nucleic acids encoding the recombinant products are those described, e.g., in WO2014055668 and U.S. Pat. No. 7,446,190, each of which is hereby incorporated by reference.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (such as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); diethylaminoethyl (DEAE)-dextran/DNA transfection (Gulick, Curr Protoc Cell Biol., Chapter 20: Unit 20.4 (2003), and strontium phosphate DNA co-precipitation (Brash et al., Mol. Cell Biol., 7: 2031-2034 (1987), each of which is hereby incorporated by reference in its entirety).

In another embodiment, the introduction and expressing of genetic material in immune cells is via a cell-delivery vehicle (e.g., cationic liposomes) or derivatized (e.g., antibody conjugated) polylysine conjugates, gramicidin S, and/or artificial viral envelopes. Such vehicles may deliver a nucleic acid that is incorporated into a plasmid, vector, or even viral DNA.

In another embodiment, the nucleic acid molecule comprising a gene of interest may be delivered into the desired cell(s) in the form of a soluble molecular complex. The complex may contain the nucleic acid releasably bound to a carrier comprised of a nucleic acid binding agent and a cell-specific binding agent which binds to a surface molecule of the desired cell(s)(e.g., T cells), and is of a size that can be subsequently internalized by the cell. Such complexes are described in U.S. Pat. No. 5,166,320 which is hereby incorporated by reference in its entirety.

Transduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, (e.g., retroviral or lentiviral, among others), transduction, transposons.

In some embodiments, the nucleic acids are introduced via a physical delivery method, such as via electroporation, particle gun, reagent-based transfection (e.g. calcium phosphate transfection), cell compression or squeezing.

In some embodiments, the spinoculation (e.g., centrifugal inoculation) of the composition containing cells, viral particles and reagent can be rotated, generally at relatively low force or speed, such as speed lower than that used to pellet the cells, such as from or from about 100 g to 3200 g (e.g., at or about or at least at or about 100 g, 200 g, 300 g, 400 g, 500 g, 1000 g, 1500 g, 2000 g, 2500 g, 3000 g or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured). In some embodiments, at least a portion of the contacting, incubating, and/or engineering of the cells, e.g., cells from an stimulated composition of CD4+ T cell or CD8+ T cells, with the virus is performed with a rotation of between about 100 g and 3200 g, 1000 g and 2000 g, 1000 g and 3200 g, 500 g and 1000 g, 400 g and 1200 g, 600 g and 800 g, 600 and 700 g, or 500 g and 700 g.

In certain embodiments, at least a portion of the engineering, transduction, and/or transfection is performed with rotation, e.g., spinoculation and/or centrifugation. In some embodiments, the rotation is performed for, for about, or for at least 5 minutes, 10 minutes, 15 minutes, 30 minutes, 60 minutes, 90 minutes, 1 hour, 2 hours, 3 hours, 4 hours, 6 hours, 8 hours, 12 hours, 24 hours, 48 hours, 72 hours, 2 days, 3 days, 4 days, 5 days, 6 days, or for at least 7 days.

In some embodiments, gene transfer is accomplished by first activating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications. In certain embodiments, the gene transfer is accomplished by first incubating the cells under activation conditions, such as by the activation unit procedure.

In some embodiments, methods for transduction or transfection are carried out by contacting one or more cells of a composition with a nucleic acid molecule encoding the recombinant protein, e.g. recombinant receptor. In some embodiments, the contacting can be effected with centrifugation, such as spinoculation (e.g. centrifugal inoculation). Such methods include any of those as described in International Publication Number WO2016/073602.

In certain embodiments, the cells are transduced in the presence of a transduction adjuvant, such as a polycations. In certain embodiments, the presence of one or more transduction adjuvants increases the efficiency of transduction. In particular embodiments, at least 25%, at least 30%, at least 40%, at least 50%, at least 60%, at least 70% at least 75%, at least 80%, at least 85%, at least 90%, at least 95%, or at least 99% of the cells that are engineered in the presence of a polycation contain or express the recombinant polynucleotide. In some embodiments, at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold more cells of a composition are engineered to contain or express the recombinant transduction adjuvants in the presence of a polycation as compared to an alternative and/or exemplary method of engineering cells without the presence of a transduction adjuvant.

In some embodiments, the cells are transfected and/or transduced in the presence of less than 100 μg/ml, less than 90 μg/ml, less than 80 μg/ml, less than 75 μg/ml, less than 70 μg/ml, less than 60 μg/ml, less than 50 μg/ml, less than 40 μg/ml, less than 30 μg/ml, less than 25 μg/ml, less than 20 μg/ml, or less than μg/ml, less than 10 μg/ml of an adjuvant. In certain embodiments, adjuvants suitable for use with the provided methods include, but are not limited to polycations, fibronectin or fibronectin-derived fragments or variants, and RetroNectin. In some embodiments, the polycation is positively-charged. In certain embodiments, the polycation reduces repulsion forces between cells and vectors, e.g., viral or non-viral vectors, and mediates contact and/or binding of the vector to the cell surface. In some embodiments, the polycation is polybrene, DEAE-dextran, protamine sulfate, poly-L-lysine, or cationic liposomes.

In some embodiments, the cells are in the presence of an activating reagent, such as described in the activation unit procedure above.

In some embodiments, engineering the cells includes a culturing, contacting, or incubation with the vector (e.g., the viral vector or the non-viral vector). In certain embodiments, the engineering includes culturing, contacting, and/or incubating the cells with the vector is performed for, for about, or for at least 4 hours, 6 hours, 8 hours, 12 hours, 16 hours, 18 hours, 24 hours, 30 hours, 36 hours, 40 hours, 48 hours, 54 hours, 60 hours, 72 hours, 1 day, 2 days, 3 days, 4 days, 5 days, 6 days, or 7 days, or more than 7 days. In particular embodiments, the engineering includes culturing, contacting, and/or incubating the cells with the vector for or for about 24 hours, 36 hours, 48 hours, 60 hours, or 72 hours, or for or for about 2 days, 3 days, 4 days, or 5 days. In some embodiments, the engineering step is performed for or for about 24 hours, 36 hours, 48 hours, 60 hours, or 72 hours. In certain embodiments, the engineering is performed for or for about 2 days.

In some embodiments, the vectors include viral vectors, e.g., retroviral or lentiviral, non-viral vectors or transposons (e.g. Sleeping Beauty transposon system), vectors derived from simian virus 40 (SV40), adenoviruses, adeno-associated virus (AAV), lentiviral vectors or retroviral vectors, such as gamma-retroviral vectors, retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV).

In some embodiments, the viral vector or the non-viral DNA contains a nucleic acid that encodes a heterologous recombinant protein. In some embodiments, the heterologous recombinant molecule is or includes a recombinant receptor (e.g., an antigen receptor), SB-transposons (e.g., for gene silencing), capsid-enclosed transposons, homologous double stranded nucleic acid (e.g., for genomic recombination) or reporter genes (e.g., fluorescent proteins such as GFP or other reporters such as luciferase).

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR) (e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV)). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the viral vector particles contain a genome derived from a retroviral genome based vector, such as derived from a lentiviral genome based vector. In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments, enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1, and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase.

The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral particles are provided at a certain ratio of copies of the viral vector particles or infectious units (IU) thereof, per total number of cells to be transduced (IU/cell). For example, in some embodiments, the viral particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10⁶ IU/mL and 5×10⁷ IU/mL, such as at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL.

In some embodiments, transduction can be achieved at a multiplicity of infection (MOI) of less than 100, such as generally less than 60, 50, 40, 30, 20, 10, 5 or less.

In some embodiments, the method involves contacting or incubating, the cells with the viral particles. In some embodiments, the contacting is for 30 minutes to 72 hours, such as 30 minute to 48 hours, 30 minutes to 24 hours or 1 hour to 24 hours, such as at least or about at least 30 minutes, 1 hour, 2 hours, 6 hours, 12 hours, 24 hours, 36 hours or more.

In certain embodiments, the input cells are treated, incubated, or contacted with particles that comprise binding molecules that bind to or recognize the recombinant receptor that is encoded by the viral DNA.

In some embodiments, the incubation of the cells with the viral vector particles results in or produces an output composition comprising cells transduced with the viral vector particles.

Inoculation Unit Operation

Once the transduction unit operation 220 is concluded, the control system initiates an inoculation unit operation 230. The inoculation method set forth below is configured to run a system with 72 conditions at a time based on the number of replicate wells used, within the transduction method. However, this can be expanded or contracted with different systems and/or system components, for example processing 192 conditions by allowing the use of a replicate number of 1 within the method. Here, transduced cells are transferred into mammalian cell deepwell plates for expansion, and the user is prompted to input a preference for forward processing or targeted inoculation TNC.

The inoculation unit operation 230 begins with worktable set up. In embodiments, the user is prompted to set up worktable 60 with DiTi, reagent troughs, cell culture media, cell counting reagent. In embodiments, the user is prompted to input inoculation mode—forward processing or targeted inoculation based on a desired TNC. In embodiments, the user is prompted to input the condition number, number of replicates (based on transduction method), incubation volume (based on the transduction inoculation volume). In embodiments, the user is prompted to input total sampling and AAA/flow cytometry volumes. In embodiments, the user can be prompted to enter these parameters in real time, or as part of a script, for example, set up by the user prior to initiation of the system 10 of the method 200 as shown in FIG. 17. In embodiments, the user is prompted to set up worktable 60 with sampling and inoculation plates based on the number of conditions input. Sampling plates include a 96-deepwell plate, a 96-well low attachment plate (cell counting), and a 96-well round bottom plate (AAA/flow cytometry).

Once the worktable 60 setup is complete, sampling is initiated by the control system 20. Plates are unlidded. If the number of replicates=2, the flexible liquid manipulation module 40 proceeds to combine replicate wells into a single well. Each combined sample well is mixed, then a total sampling volume is aspirated per sample and dispensed into the 96-deepwell plate. Plates are relidded. The dispensed sampling volume is then mixed, and aliquot into the cell counting and analytical sample plates (e.g., AAA/flow cytometry plates). In embodiments, the cell counting reagent is then dispensed into the low attachment cell counting plates according the number of conditions input. In certain embodiments, the cell counts for the sampling plates are automatically read by the cell counting module 75. In other embodiments, the sampling plates are then brought to the front of the worktable 60 for user reachability, then removed from the worktable 60 by a user for manual cell counting. Cell concentration measurements are obtained by the system controller 20, either automatically from the cell counting module 75 or as manually entered by a user.

Once the sampling is complete, inoculation is initiated by the control system 20. If targeting inoculation is selected, the user is prompted to input the measured VCC, desired inoculation TNC and VCC. Based on the current VCC, the required cell volume to reach the target TNC and the required balance media volume to reach the desired inoculation VCC are calculated. If forward processing is selected, the user is not prompted to input their measured VCC values, and can directly proceed with inoculation. Based on the user input, the user is then prompted to place “n” number of 24-deepwell expansion plates according the number of conditions input. The container manipulation module 50 unlids both the expansion plates and the expansion plates, then dispenses either the required expanded cell material to reach the inoculation TNC or the entire expansion plate contents according to the user input. The flexible liquid manipulation module 40 follows by dispensing balance cell culture media into each well of the 24-deepwell expansion plate per condition to reach a final inoculation volume of 3 mL. The container manipulation module 50, using eccentric fingers 52, lids all 24-deepwell expansion plates on the worktable 60. In embodiments, the plates are automatically transferred to a mammalian cell incubator for inoculation. In embodiments, the all remaining labware is automatically removed from the worktable. In embodiments, a user is prompted to place plates into the incubator for inoculation. In embodiments a user is prompted remove all remaining labware from the worktable. The user is then prompted to remove all remaining labware from the worktable 60.

Expansion Unit Operation

With reference to FIG. 17, once the inoculation unit operation 230 is concluded, the control system initiates an expansion unit operation 240. In embodiments, the inoculation unit operation 230 and the expansion unit operation 240 are separated by an incubation period, for example between about 1 day and about 3 days based on the scale down process being performed. The expansion unit operation 240 begins with worktable set up. In embodiments, the user is prompted to setup worktable 60 with DiTi, reagent troughs, cell culture media, cell counting reagent. User is prompt to place balance 24-deepwell expansion plates onto the worktable 60. 24-deepwell balance expansion plates are used for cell centrifugation. In embodiments, the user is prompted to input condition number and expansion volume. User is prompt to input total sampling and AAA/flow cytometry volumes. In embodiments, the user is prompted to setup worktable 60 with sampling and “n” number of 24-deepwell expansion plates based on the number of conditions input. Sampling plates include a 96-deepwell plate, a 96-well low attachment plate (cell counting), and a 96-well round bottom plate (AAA/flow cytometry). In some examples, one or more of the above-mentioned steps may be performed automatically, without departing from the scope of this disclosure.

Once the worktable 60 setup is complete, sampling is initiated by the control system 20. Expansion plates are unlidded, then a total sample volume is aspirated per sample and dispensed into the 96-deepwell plate. Expansion plates are relidded using the container manipulation module 50. The dispensed sampling volume is then mixed, and aliquot into the cell counting and AAA/flow cytometry plates. The cell counting reagent is then dispensed into the low attachment cell counting plates according the number of conditions input. In certain embodiments, the cell counts for the sampling plates are automatically read by the cell counting module 75. In other embodiments, the sampling plates are then brought to the front of the worktable 60 for user reachability, then removed from the worktable 60 by a user for manual cell counting. Cell concentration measurements are obtained by the system controller 20, either automatically from the cell counting module 75 or as manually entered by a user. The current expansion method uses the measured VCC per condition so that the method is allowed to proceed independent of the cell counts (e.g., the system may forward process independent of the cell counts).

Once the sampling is complete, mock perfusion/cell culture media exchange is initiated by the control system 20. The container manipulation module 50, using centric fingers 54, transports the expansion plates and its balance plate (only with odd number of 24-deepwell centrifuge plates) vertically into the robotic centrifuge 65. Post centrifugation, the container manipulation module 50 returns the 24-deepwell centrifuge plates back to the worktable 60. Using FES, the container manipulation module 50 replaces the centric finger 54 with an eccentric finger 52, and unlids the expansion plates. The flexible liquid manipulation module 40, such as FCA, may then remove a fraction of the expansion volume cell culture supernatant without dislodging the cell pellet below. This is performed per well according to the number of conditions input. The flexible liquid manipulation module 40, such as FCA follows by dispensing fresh cell culture media into each well of the 24-deepwell expansion plate per condition to reach the final expansion volume as input. The container manipulation module 50, using eccentric fingers 52, lids all 24-deepwell expansion plates on the worktable 60. In embodiments, the plates are automatically transferred to a mammalian cell incubator. In embodiments, the all remaining labware is automatically removed from the worktable. In embodiments, a user is prompted to place plates into the incubator for. In embodiments a user is prompted remove all remaining labware from the worktable. The user is then prompted to remove all remaining labware from the worktable 60.

The expansion method set forth below is configured to run a system with up to 192 conditions at a time. This method performs sampling and mock perfusion steps. In certain embodiments, mock perfusion is executed by centrifugation of the expansion plate, followed by a media exchange. In certain embodiments, a cell passaging strategy that will be defined based on the VCC per condition well.

In some embodiments, the cells are cultivated under conditions that promote proliferation and/or expansion. In some embodiments, such conditions may be designed to induce proliferation, expansion, activation, and/or survival of cells in the population. In particular embodiments, the activation conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the cells.

In some embodiments, the cultivation is performed under conditions that generally include a temperature suitable for the growth of primary immune cells, such as human T lymphocytes, for example, at least about 25 degrees Celsius, generally at least about 30 degrees, and generally at or about 37 degrees Celsius. In some embodiments, the composition of enriched T cells is incubated at a temperature of 25 to 38° C., such as 30 to 37° C., for example at or about 37° C.±2° C. In some embodiments, the incubation is carried out for a time period until the culture, e.g., cultivation or expansion, results in a desired or threshold density, number or dose of cells. In some embodiments, the incubation is greater than or greater than about or is for about or 24 hours, 48 hours, 72 hours, 96 hours, 5 days, 6 days, 7 days, 8 days, 9 days or more.

In some embodiments, the activation reagent is removed and/or separated from the cells prior to the cultivation. In some embodiments, the activation reagent is an activation reagent that is described in the activation unit procedure. In some embodiments, the activation reagent is removed and/or separated from the cells after or during the cultivation.

In particular embodiments, the cells are cultivated in the presence of one or more cytokines. In certain embodiments, the one or more cytokines are recombinant cytokines. In particular embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes recombinant IL-2.

In some embodiments, the cultivation is performed for the amount of time required for the cells to achieve a threshold amount, density, and/or expansion. In some embodiments, the cultivation is performed for or for about, or for less than, 6 hours, 12 hours, 18 hours, 24 hours, 36 hours, 48 hours, 60 hours, 72 hours, 2 days, 3 days 4 days, 5 days, 6 days, 7 days, 8 days, 9 days, 10 days, 1 week, 2 weeks, 3 weeks, or 4 weeks.

Debeading Unit Operation

With reference to FIG. 17, once the expansion unit operation 240 is concluded, the control system initiates a debeading unit operation 250. The debeading unit operation 250 begins with worktable set up. In embodiments, a user is prompted to setup worktable 60 with DiTi, reagent troughs, cell culture media, cell counting reagent. In embodiments, a user is prompted to place skirted magnets onto the worktable 60. In embodiments, a user is prompted to input condition number and expansion volume. In embodiments, a user is prompted to input total sampling and analytical sample volumes, such as AAA/flow cytometry volumes. In embodiments, a user is also prompted to input if a second sampling step is desired after debeading. In embodiments, a user is prompted to setup worktable 60 with sampling and “n” number of 24-deepwell expansion plates based on the number of conditions input. Sampling plates include a 96-deepwell plate, a 96-well low attachment plate (cell counting), and a 96-well round bottom plate (analytical sample plate, e.g., AAA/flow cytometry sample plate). In some examples, one or more of the above-mentioned steps may be performed automatically, without departing from the scope of this disclosure.

Once the worktable 60 setup module is complete, sampling is initiated by the control system 20. Expansion plates are unlidded, then a total sample volume is aspirated per sample and dispensed into the 96-deepwell plate. Expansion plates are relidded. The dispensed sampling volume is then mixed, and aliquot into the cell counting and AAA/flow cytometry plates. The cell counting reagent is then dispensed into the low attachment cell counting plates according the number of conditions input. In certain embodiments, the cell counts for the sampling plates are automatically read by the cell counting module 75. In other embodiments, the sampling plates are then brought to the front of the worktable 60 for user reachability, then removed from the worktable 60 by a user for manual cell counting. Cell concentration measurements are obtained by the system controller 20, either automatically from the cell counting module 75 or as manually entered by a user. The debeading method uses the measured VCC per condition as FIO, therefore the method is allowed to proceed independent of the cell counts.

Once the sampling is complete, debeading is initiated by the control system 20. In embodiments, a user is prompted to place “n” number of fresh 24-deepwell expansion plates onto the worktable 60 based on the number of conditions input. The container manipulation module 50, using eccentric fingers 52, unlids the original and new 24-deepwell expansion plates. Using FES, the container manipulation module 50 replaces the eccentric fingers 52 for centric, and proceeds to place the original expansion plate onto the skirted magnet. The method pauses for 5 minutes to allow for debeading to occur. The flexible liquid manipulation module 40 then aspirates the debeaded product and dispenses it into the fresh 24-deepwell expansion plate. Aspiration occurs with an x offset, as to not disrupt the bead pellet below. The container manipulation module 50, such as an RGA, using eccentric fingers 52, relids all 24-deepwell expansion plates.

Once the debeading is complete, a second sampling is initiated by the control system 20. The user is then prompted to remove all labware from the worktable 60 and prepare for harvest or inoculation.

The debeading unit procedure 250 is applicable for both processing T cells prior to inoculation and T cells prior to harvest. Here, 24-deepwell expansion plates will be placed on the deck, sampled, counted, placed on an on-deck magnet and transferred to a 24 deep well expansion plate. Debeading is stand-alone method from harvest or inoculation due to the differences in the required worktable 60 for method execution. This method allows for 2 sampling steps. One prior to debeading, and the other directly after. The second sampling step serves two purposes. The first is to allow user determination of debeading cell yield, the other is to provide updated cell measurements post-debeading that will be used to inform the harvest method processing.

Harvest Unit Operation

Once the debeading unit operation 250 is concluded, the control system initiates a harvest unit operation 260.

The harvest unit operation 260 begins with worktable set up. In embodiments, the user is prompt to setup worktable 60 with DiTi, reagent troughs, and cryopreservation media. In some embodiments, the worktable also includes a cooling device 61 to cool reagents prior to use in the described methods. Cooling device 61 may be a thermoelectric cooler, a cooler that relies on a refrigerant, a cooler that relies on an insulating gel or other insulating material, a cooler for use with one or more of liquid nitrogen, dry ice and/or water ice, among others. In embodiments, the user is prompted to place balance 24-deepwell expansion plates onto the worktable 60. 24-deepwell expansion balance plates are used for cell centrifugation. In embodiments, the user is prompted to input condition number, expansion culture volume and the debeading sampling volume. In embodiments, the user is prompted to input the VCC and number of vials to be cryopreserved per condition into a harvest excel workbook. Based on the current VCC, and the desired VCC and TNC per cryovial, the required debeaded product volume and cryopreservation media is calculated. The vial number per condition will serve as replicate vials for analytical testing. In embodiments, the user is prompted to setup worktable 60 by adding a set number of uncapped cryovial according to the inputs. In embodiments, the user is then prompted to place their debeaded product plate onto the worktable 60.

Once the worktable 60 setup is complete, cryopreservation of cell samples is initiated by the control system 20. The container manipulation module 50 with centric fingers 54, transports the expansion plate and its balance plate vertically into the robotic centrifuge 65. Post centrifugation, the debeaded product plate is returned to the worktable 60. The container manipulation module 50 unlids the debeaded product plate, followed by flexible liquid manipulation module 40, such as FCA aspiration of the supernatants per condition well. Aspiration is performed at reduced speed with a well offset to ensure that the cell pellet is not disturbed. The flexible liquid manipulation module 40 dispenses the balance cryomedia volume required to reach the desired cryopreserved VCC as input. The flexible liquid manipulation module 40, such as FCA follows by dispensing a variable volume of cryopreservation media into each well of the debeaded product plate according to the number of conditions input. The flexible liquid manipulation module 40, such as FCA then performs 2 mixing cycles to ensure complete mixing of the cryopreservation media and the pelleted cells. The flexible liquid manipulation module 40 then aspirates the desired TNC per cryovial required to meet the desired VCC input. For each condition, the cells are dispensed into cryovials, based on the number of cryovials allocated per condition. The container manipulation module 50 using tube fingers 56, caps each of the cryovials based on the total cryovials required as input. In embodiments, the user is then prompted to place cryovials into a cell freezing container or a controlled rate freezer (CRF). The container manipulation module 50, with eccentric fingers 52 relids the debeaded product plate and prompts the user to remove all labware from the worktable 60.

Harvest method described can currently process up to 24 cell culture conditions and cryopreserve up to 96 vials at a time. Based off the VCC measured during the debeading method, the user is able to cryopreserved cells at a desired cell density.

Recombinant Proteins

In embodiments the methods and systems disclosed herein are used to produce cells, such as T cell, for example CD4+ and/or CD8+ T cells that contain or express, or are engineered to contain or express, a recombinant protein, such as a recombinant receptor (e.g., a chimeric antigen receptor (CAR), or a T cell receptor (TCR)). In certain embodiments, the methods provided herein produce and/or a capable of producing cells, or populations or compositions containing and/or enriched for cells, that are engineered to express or contain a recombinant protein.

The cells generally express recombinant receptors, such as antigen receptors including functional non-TCR antigen receptors (e.g., chimeric antigen receptors (CARs)), and other antigen-binding receptors such as transgenic T cell receptors (TCRs). Also among the receptors are other chimeric receptors

Chimeric Antigen Receptors

In some embodiments of the provided methods and uses, chimeric receptors, such as a chimeric antigen receptors, contain one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

The chimeric receptors, such as CARs, generally include an extracellular antigen binding domain, such as a portion of an antibody molecule, generally a variable heavy (VH) chain region and/or variable light (VL) chain region of the antibody (e.g., an scFv antibody fragment).

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv.

In some embodiments, the antibody portion of the recombinant receptor (e.g., CAR), further includes at least a portion of an immunoglobulin constant region, such as a hinge region (e.g., an IgG4 hinge region), and/or a CH1/CL and/or Fc region. In some embodiments, the constant region or portion is of a human IgG, such as IgG4 or IgG1. In some aspects, the portion of the constant region serves as a spacer region between the antigen-recognition component (e.g., scFv), and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In some embodiments, the constant region or portion is of a human IgG, such as IgG4 or IgG1.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an immunoreceptor tyrosine-based activation motif (ITAM). For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor (e.g., the CAR), generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs that are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity (e.g., CD3 zeta chain). Thus, in some aspects, the antigen-binding portion is linked to one or more cell signaling modules. In some embodiments, cell signaling modules include CD3 transmembrane domain, CD3 intracellular signaling domains, and/or other CD3 transmembrane domains. In some embodiments, the receptor (e.g., CAR), further includes a portion of one or more additional molecules such as Fc receptor, CD8, CD4, CD25, or CD16. For example, in some aspects, the CAR or other chimeric receptor includes a chimeric molecule between CD3-zeta (CD3-) or Fc receptor and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell (e.g., T cell engineered to express the CAR). For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand (e.g., to reduce off-target effects).

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more (e.g., two or more), costimulatory domains and an activation domain (e.g., primary activation domain), in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

In some embodiments, the marker is a molecule (e.g., cell surface protein), not naturally found on T cells or not naturally found on the surface of T cells, or a portion thereof.

In some embodiments, the molecule is a non-self molecule (e.g., non-self protein), i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody (e.g., antibody fragment), a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge (e.g., an IgG4 hinge), such as a hinge-only spacer.

In some embodiments, the transmembrane domain of the recombinant receptor (e.g., the CAR), is or includes a transmembrane domain of human CD28 (e.g., Accession No. P01747.1) or variant thereof. In some embodiments, the intracellular signaling component(s) of the recombinant receptor (e.g., the CAR), contains an intracellular costimulatory signaling domain of human CD28 or a functional variant or portion thereof, such as a domain with an LL to GG substitution at positions 186-187 of a native CD28 protein. In some embodiments, the intracellular domain comprises an intracellular costimulatory signaling domain of 4-1BB (Accession No. Q07011.1) or functional variant or portion thereof.

In some embodiments, the intracellular signaling domain of the recombinant receptor (e.g. the CAR), comprises a human CD3 zeta stimulatory signaling domain or functional variant thereof, such as a 112 AA cytoplasmic domain of isoform 3 of human CD3 (Accession No.: P20963.2) or a CD3 zeta signaling domain as described in U.S. Pat. No. 7,446,190 or 8,911,993.

In some aspects, the spacer contains only a hinge region of an IgG, such as only a hinge of IgG4 or IgG1, such as the hinge only spacer. In other embodiments, the spacer is or contains an Ig hinge (e.g., an IgG4-derived hinge), optionally linked to a CH2 and/or CH3 domains. In some embodiments, the spacer is an Ig hinge (e.g., an IgG4 hinge), linked to CH2 and CH3 domains. In some embodiments, the spacer is an Ig hinge (e.g., an IgG4 hinge), linked to a CH3 domain only. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, nucleic acid molecules encoding such CAR constructs further includes a sequence encoding a T2A ribosomal skip element and/or a tEGFR sequence (e.g., downstream of the sequence encoding the CAR). In some embodiments, T cells expressing an antigen receptor (e.g., CAR) can also be generated to express a truncated EGFR (EGFRt) as a non-immunogenic selection epitope (e.g., by introduction of a construct encoding the CAR and EGFRt separated by a T2A ribosome switch to express two proteins from the same construct), which then can be used as a marker to detect such cells (see, for example, U.S. Pat. No. 8,802,374). In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus, equine rhinitis A virus, Thosea asigna virus, and porcine teschovirus-1 as described in U.S. Patent Publication No. 20070116690.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

TCRs

In some embodiments, engineered cells, such as T cells, are provided that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains one or more variable α and β chains expressed as part of a complex with CD3 chain molecules. A minority of T cells express an alternate receptor, formed by variable γ and δ chains. Within these chains are complementary determining regions (CDRs) which determine the antigen bound to an MHC molecule to which the TCR will bind. TCRs activate the T cells in which they reside, upon recognition of the antigen, leading to a plethora of immune responses. Typically, TCRs are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the form or form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable chain and variable chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the -chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain(s). For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α and/or β-chain constant domain or C, typically positions 117 to 259 of the chain based on Kabat numbering or chain constant domain or C, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the constant domains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ε chains) contain one or more immunoreceptor tyrosine-based activation motif or ITAM that are involved in the signaling capacity of the TCR complex.

In some embodiments, the TCR may be a heterodimer of two chains and/or it may be a single chain TCR construct. In some embodiments, the TCR is a heterodimer containing two separate chains that are linked, such as by a disulfide bond or disulfide bonds.

In some embodiments, the TCR can be generated from a known TCR sequence(s), such as sequences of variable (V) chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of V chains from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ T cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of V chain sequences, such as by RT-PCR in samples, such as T cells, obtained from humans. In some embodiments, scTv libraries can be assembled from naïve V chain libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess cytotoxic T lymphocyte (CTL) activity against the peptide. In some aspects, TCRs (e.g., present on the antigen-specific T cells), may be selected, such as by binding activity (e.g., particular affinity or avidity for the antigen).

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MEW class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. Immunoinformatics Methods in Molecular Biology, vol 409(1): 75-93 2007)

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments, a dTCR contains a first polypeptide wherein a sequence corresponding to a TCR chain variable region sequence is fused to the N terminus of a sequence corresponding to a TCR chain constant region extracellular sequence, and a second polypeptide wherein a sequence corresponding to a TCR chain variable region sequence is fused to the N terminus a sequence corresponding to a TCR chain constant region extracellular sequence, the first and second polypeptides being linked by a disulfide bond. In some embodiments, the bond can correspond to the native inter-chain disulfide bond present in native dimeric TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR chain containing a variable domain, a constant domain and a first dimerization motif attached to the C-terminus of the constant domain, and a TCR chain comprising a variable domain, a constant domain and a first dimerization motif attached to the C-terminus of the constant domain, wherein the first and second dimerization motifs easily interact to form a covalent bond between an amino acid in the first dimerization motif and an amino acid in the second dimerization motif linking the TCR chain and TCR chain together.

In some embodiments, the TCR is a scTCR. Typically, a scTCR can be generated using methods known, see e.g., Soo Hoo, W. F. et al. PNAS (USA) 89, 4759 (1992); Wülfing, and PlUckthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCR variable domain covalently linked to a TCR variable domain via a peptide linker (see e.g., International published PCT No. WO99/18129).

In some embodiments, a scTCR contains a first segment constituted by an amino acid sequence corresponding to a TCR chain variable region, a second segment constituted by an amino acid sequence corresponding to a TCR chain variable region sequence fused to the N terminus of an amino acid sequence corresponding to a TCR chain constant domain extracellular sequence, and a linker sequence linking the C terminus of the first segment to the N terminus of the second segment.

In some embodiments, a scTCR contains a first segment constituted by an chain variable region sequence fused to the N terminus of an chain extracellular constant domain sequence, and a second segment constituted by a chain variable region sequence fused to the N terminus of a sequence chain extracellular constant and transmembrane sequence, and, optionally, a linker sequence linking the C terminus of the first segment to the N terminus of the second segment.

In some embodiments, a scTCR contains a first segment constituted by a TCR chain variable region sequence fused to the N terminus of a chain extracellular constant domain sequence, and a second segment constituted by an chain variable region sequence fused to the N terminus of a sequence chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids.

In some embodiments, the scTCR contains a covalent disulfide bond linking a residue of the immunoglobulin region of the constant domain of the chain to a residue of the immunoglobulin region of the constant domain of the chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine.

Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10⁻⁵ and 10⁻¹² M and all individual values and ranges therein. In some embodiments, the target antigen is an MHC-peptide complex or ligand.

In some embodiments, nucleic acid or nucleic acids encoding a TCR or portions thereof, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as G10, GT11, ZapII (Stratagene), EMBL4, and NM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a vector encoding a TCR, the α and β chains (for example) are PCR amplified from total cDNA isolated from a T cell clone expressing the TCR of interest and cloned into an expression vector. In some embodiments, the α and β chains (for example) are cloned into the same vector. In some embodiments, the α and β chains are cloned into different vectors. In some embodiments, the generated and chains are incorporated into a retroviral, e.g., lentiviral, vector.

Liquid Class Determination

A liquid class is a collection of parameters required for pipetting liquids. There are two sets of parameters in the disclosed system 10 associated with liquid transfer. These are pipetting parameters and calibration parameters. Pipetting parameters are more related to precision than accuracy and include factors such as aspiration and dispensing speed, air gap or liquid contact during dispensing. Calibration parameters are more related to accuracy than precision, and define the slope and offset of the calibration curve for a specific liquid class. Instead of optimizing both sets of parameters for every new liquid class, screened predefined liquid classes to identity a default class that had optimal parameters for a precise pipetting are determined, then the calibration settings are adjusted to improve pipetting accuracy.

The values of the pipetting and calibration parameters are dependent on the physical characteristics of the liquids. These are defined per liquid type and pipette mode (i.e, single vs multipipette; free vs wet contact dispense etc). One liquid class covers the whole volume range for both a FCA and MCA 384. Within each liquid class, subclasses were created. Subclasses are defined based on the arm and tip type. It was within these subclasses that pipetting and calibration parameters are defined.

A gravimetric approach was employed in developing liquid classes. The Weigh Module (0.01 mg resolution) from Mettler Toledo was used as an on-deck balance. This balance was integrated with the control system 20 to allow for automated zero and measure commands. Liquid class screening, optimization and confirmations were performed in an automated manner with independent methods for each step.

Liquid classes enable pipetting automation by translating manual pipetting steps into an automated process. Typically, pipetting is affected by several calibration parameters such as volume, temperature, density, and viscosity, as well as several pipetting parameters such as airgaps, delays, and pipetting speeds. More viscous liquids, such as DMSO (a liquid used in the cryopreservation of cells), often require slower pipetting speeds and delays to improve the accuracy and precision of pipetting compared to less viscous liquids, such as water, which generally can perform with high precision and accuracy at faster speeds and shorter delays. Inaccurate pipetting of multiple solutions can have compounded effects on the overall process impacting the analysis of results. Within the automated scale down model methods, every aspiration and dispense liquid transfer step requires a developed liquid class. This liquid class defines how that particular liquid will be aspirated/dispensed with each tip type and how it responds to errors during method execution. The liquid class will also be used for informing the appropriate working volume ranges for each pipette tip type for a given liquid, based on the measured accuracy and precision.

Liquid Class Components and Parameters

There are four main components used to develop a liquid class. These include the easy control, detection and positioning, formulas, and microscript commands.

Easy Control

The easy control component is a graphical editor that allows for easy control of critical parameters for aspiration and dispense. Within easy control, parameters such as volume can be adjusted to visualize potential airgaps, delays, and speeds that may be needed to pipet a certain volume with pipette tip type.

Detecting and Positioning

The detection and positioning components allow the user to set parameters for capacitive liquid level detection (cLLD), tracking options, error handling, and retract properties for aspiration and dispense. cLLD is a means by which the liquid handler senses the presence and height of liquid in the labware. Using a grounded worktable 60 and conductive pipetting tips, the liquid handler responds to the capacitive change at the air/liquid interface. cLLD is turned on for all automated scale down model applications. cLLD begins at Zₛₜₐᵣₜ and proceeds till Zₘₐₓ. Zₜᵣₐᵥₑₗ indicates the minimum distance from the deck a pipette tip can come near the labware when in free motion. Zₛₜₐᵣₜ is the distance inside the labware over the liquid level during the beginning of an aspirations step. Zₛₜₐᵣₜ is defined by labware definitions. Zₘₐₓ is set so that the pipette tip does not crash to the bottom of the labware and is also defined by labware definitions. Per liquid class, if cLLD is selected, the measured sensitivity group, and tip submersion depth is input.

As a liquid is aspirated, if cLLD and tracking is turned on, the pipette tip will enter the liquid and proceed to the set submerge depth. It will then move downwards at the rate of liquid aspiration in order to maintain that submersion depth as liquid is aspirated. Tracking is turned on for all aspiration steps within the automated scale down model methods. Retract properties and supervision allow the system to monitor the exit of the pipette tip from the liquid and informs the user if errors were experienced during the tip retraction. The error handing section defines how the liquid handler responds to errors experienced during an experiment. This is determined per liquid class. Typically “user prompt” is selected per liquid class, whereby the user is allowed to see the error message during method execution and can respond directly.

Formulas

The formulas provide a way to enter liquid handling parameters as a function. These formulas can be edited for both aspiration and dispense with fixed values or pipetting volume dependent formulas. Different volumes of liquid will have different offsets for accuracy of pipetting specific to a liquid class. These offsets are adjusted during liquid class development.

Microscript

The microscript is the underlying sequence of actions during aspiration, dispense, and mix. In the aspiration script, a few variables are set for volume (includes the offset from the accuracy adjustment), acceleration and deceleration. The three way check valve is turned to the pipette tip position to allow liquid to be aspirated. An initial leading airgap is aspirated and then liquid is pre-wetted to a set number of cycles set in the variables section. The software checks if cLLD is turned on or off to determine its mode of aspiration. After aspirating the liquid with or without cLLD, a trailing airgap is aspirated. During dispense of the liquid, the script sets the variable for volume (includes the offset from the accuracy adjustment). The software checks to see if multi-pipetting is turned on or off. It then moves the three way check valve back to the pipette tip position to allow liquid to be dispensed. The software once again checks to see if cLLD is turned on or off. It then dispenses the liquid with the appropriate settings (with or without multi-pipetting and with or without cLLD). If a delay has been set, the pipette tip will wait a set amount of time before retracting from the labware. It will then move to a set z-position. If a trailing airgap has been set it will then aspirate air before moving the pipette tip.

Cell Culture Test Liquids

Cell Culture Liquids

Media

Basal Media

Complete

Cell solutions

Cells+Media

Cells+CryoStor/PlasmaLyte/HSA

Cryostor/PlasmaLyte/HSA

Arms Types

FCA

MCA384

FCA Subclasses

5000 μL DiTi

1000 μL DiTi

200 μL DiTi

50 μL DiTi

10 μL DiTi

MCA Subclasses

50 μL DiTi

150 μL DiTi

Contact Dispense

Free

Contact

Dispense Type

Single

Multi

Mixing

Media

Cells+Media

Cells+ CryoStor/PlasmaLyte/HAS

Methodology

A Liquid Class Workbook was Created for Each Test Liquid.

Using the Densito 30PX, the density of the test liquid was measured and recorded within the “Liquid Details” tab in the Liquid Class Workbook. All additional 30PX liquid details were reported in the “Liquid Details” tab.

The cLLD sensitivity group (low, medium, high) was verified for each test liquid using the detect sensitivity command. The sensitivity group was recorded within the “Liquid Details” tab in the Liquid Class Workbook.

Ideally, a free/singe-dispense default liquid class was to be identified for every test liquid. This default liquid class was also used as the default for both multi-dispense and MCA liquid classes. All default liquid classes were then optimized to improve pipetting accuracy. If the free/singe-dispense default liquid class yielded low precision for multi-dispense and MCA liquid classes, pipetting parameters were optimized for the test liquid.

Flexible Channel Arm Liquid Classes

Free Single Dispensing

Liquid Class Screen

New Cell Culture Liquids

Every new liquid was screened against 5 established liquid classes. 500 μL was used as a screening volume for determining a default liquid class. The minimum, maximum, mean, accuracy and precision of the dispense volume was calculated per each liquid class type tested. The default class was determined as the class having the highest precision (% CV) and accuracy (% DEV). For highly viscous liquids, the liquid was to be additionally screened against established contact liquid classes. If contact was used, a separate contact liquid class was created. If all liquid classes yield poor accuracy and precision, pipetting parameters were adjusted. Volume measurements were attained with all 8 FCA channels, however, all statistics were calculated with 1.25 mL syringes alone. To prevent evaporation, one pipette tip was used per pipetting cycle. Liquid Class Screening for non-viscous liquids were performed in free and single dispense modes.

“Known” cell culture liquids

Liquids that bare similar physical properties to a previously established liquid class may skip screening steps and can adopt the established liquid class as its default (starting) liquid class. i.e. media formulations

Liquid Class Optimization

To optimize a default liquid class for a test liquid, a duplicate of the default liquid class attained from the liquid class screening step was created. 6 test volumes were assessed per DiTi subclass to create an optimized test liquid class with high precision and accuracy.

10-1000 μL DiTi Test Volumes FCA 1-4

Liquid class optimization was executed with 5 liquid subclasses (FCA 1-5), as shown in Table 1. Within each subclass, 6 volumes were tested to assess pipetting accuracy. Liquid subclasses and test volumes were reported in the “Test Volume” tab in the Liquid Class Workbook. Each test volume had 8 replicates, with 1 replicate per channel. The measured volume dispensed from each channel was reported in the “Opt FCA” tabs. Volume measurements were attained with all 8 FCA channels, however, all statistics were calculated with 1.25 mL syringes alone. The minimum, maximum, mean, accuracy (% DEV) and precision (% CV) of the dispensed volume was calculated per test volume. If the % DEVs and % CVs were within the acceptance criteria, confirmation runs were initiated. If they were above the acceptance criteria, additional iterations were performed. To prevent evaporation, one pipette tip was used per pipetting cycle.

5000 μL DiTi Test Volumes FCA 5

Each test volume had 6 replicates, with 3 replicates per channel. The measured volume dispensed from each channel was reported in the “Opt FCA 5” tab. The minimum, maximum, mean, accuracy (% DEV) and precision (% CV) of the dispensed volume was calculated per test volume. If the % DEVs and % CVs were within the acceptance criteria, confirmation runs can be initiated. If they were above the acceptance criteria, additional iterations were performed.

TABLE 1
Flexible Channel Arm: Single Dispense

| DiTi | Sub- | | | | | | |
|---|---|---|---|---|---|---|---|
| Subclasses | classes | Test Volumes (μL) | | | | | |
| 10 μL DiTi | FCA 1 | 0.25 | 1 | 2.5 | 5 | 7.5 | 10 |
| 50 μL DiTi | FCA 2 | 0.5 | 1 | 2 | 10 | 20 | 50 |
| 200 μL DiTi | FCA 3 | 2 | 5 | 10 | 50 | 100 | 200 |
| 1000 μL DiTi | FCA 4 | 5 | 10 | 50 | 100 | 500 | 1000 |
| 5000 μL DiTi | FCA 5 | 300 | 500 | 100 | 1500 | 2500 | 4700 |

Confirmation Runs

Experiments and data package remained the same as Liquid Class Optimization steps, however, each volume was tested 3× with the finalized accuracy adjustment. The final mean, CV and % DEV were calculated from 18 measurements for both 1.25 mL (3×6 1.25 mL syringes) and 5 mL (9×2 5 mL syringes) syringes. If % CVs and % DEVs remained within the acceptance criteria, the liquid class was completed.

Multi Dispensing

Liquid Class Optimization

For Multi-dispense liquid classes, the screening step was skipped and optimization proceeded directly (if good precision was maintained). Each test multi-dispense liquid class was created as a duplicate of the default multi-dispense liquid class already optimized for the test liquid (i.e. the “multi” version of the default liquid class). 3 liquid subclasses were built for each multi-dispense liquid class (FCA 3-5M), as shown in Table 2. Unlike single dispensing liquid classes, only one channel was used for multi-dispensing liquid classes.

For each subclass, 6 volumes were tested to assess pipetting accuracy. Liquid subclasses and test volumes were reported in Table 2 and on the “Test Volume” tab in the Liquid Class Workbook. Each test volume had 8 replicates, with 8 replicated performed with 1 channel. The measured volume dispensed from each dispense was reported in the “Opt FCA M” tabs. The minimum, maximum, mean, accuracy (% DEV) and precision (% CV) of the dispensed volume was calculated per test volume. If the % DEVs and % CVs were within the acceptance criteria, confirmation runs can be initiated. If they were above the acceptance criteria, additional iterations can be performed.

TABLE 2
Flexible Channel Arm: Multi-Dispense

| DiTi | Sub- | | | | | | |
|---|---|---|---|---|---|---|---|
| Subclasses | classes | Test Volumes | | | | | |
| 200 μL DiTi | FCA 3 | 1 | 5 | 10 | 20 | 35 | 50 |
| 1000 μL DiTi | FCA 4 | 10 | 25 | 50 | 100 | 200 | 400 |
| 5000 μL DiTi | FCA 5 | 50 | 100 | 200 | 500 | 1000 | 2000 |

Confirmation Runs

Experiments and data package remained the same as Liquid Class Optimization steps, however, each volume was tested 3× with the finalized accuracy adjustment. The final mean, CV and % DEV were calculated from 24 measurements for both 1.25 mL and 5 mL syringes. If CV and DEV remained within the acceptance criteria, the liquid class was completed.

Additional Equipment

TABLE 3
Exemplary equipment for gravimetric liquid class development

| Item | Manufacturer | Model Number | Part Number |
|---|---|---|---|
| Balance | Mettler Toledo | Weigh Module | 111210023 |
| | | WXSS205S/15 | |
| Density Meter | Mettler Toledo | Densito 30PX | 51324450 |
| Grid Segment, 6 Grids | Tecan | N/A | 30042701 |
| Mounting Plate | Tecan | N/A | 30042796 |

Sequences

Sequences

| # | SEQUENCE | ANNOTATION |
|---|---|---|
| 1 | DPSKDSKAQVSAAEAGITGT | Streptavidin |
| | WYNQLGSTFIVTAGADGALT | Species: Streptomyces |
| | GTYESAVGNAESRYVLTGRY | avidinii |
| | DSAPATDGSGTALGWTVAWK | UniProt No. P22629 |
| | NNYRNAHSATTWSGQYVGGA | |
| | EARINTQWLLTSGTTEANAW | |
| | KSTLVGHDTFTKVKPSAASI | |
| | DAAKKAGVNNGNPLDAVQQ | |
| 2 | EAGITGTWYNQLGSTFIVTA | Minimal streptavidin |
| | GADGALTGTYESAVGNAESR | Species: Streptomyces |
| | YVLTGRYDSAPATDGSGTAL | avidinii |
| | GWTVAWKNNYRNAHSATTWS | |
| | GQYVGGAEARINTQWLLTSG | |
| | TTEANAWKSTLVGHDTFTKV | |
| | KPSAAS | |
| 3 | His-Pro-Gln-Phe | Streptavidin-binding |
| | | peptide |
| 4 | Oaa-Xaa-His-Pro-Gln- | Streptavidin-binding |
| | Phe-Yaa-Zaa | peptide |
| | | Oaa is Trp, Lys or |
| | | Arg; |
| | | Xaa is any amino acid; |
| | | Yaa is Gly or Glu |
| | | Zaa is Gly, Lys or Arg |
| 5 | -Trp-Xaa-His-Pro- | Streptavidin-binding |
| | Gln-Phe-Yaa-Zaa- | peptide |
| | | Xaa is any amino acid; |
| | | Yaa is Gly or Glu |
| | | Zaa is Gly, Lys or Arg |
| 6 | Trp-Arg-His-Pro-Gln- | Streptavidin binding |
| | Phe-Gly-Gly | peptide, Strep-tag® |
| 7 | WSHPQFEK | Strep-tag® II |
| 8 | Trp-Ser-His-Pro-Gln- | Sequential modules |
| | Phe-Glu-Lys-(Xaa)n- | of streptavidin- |
| | PTrp-Ser-His-Pro-Gln- | binding peptide |
| | he-Glu-Lys- | Xaa is any amino acid; |
| | | n is either 8 or 12 |
| 9 | Trp-Ser-His-Pro-Gln- | Sequential modules of |
| | Phe-Glu-Lys-(GlyGly | streptavidin-binding |
| | GlySer)n-Trp-Ser-His- | peptide |
| | Pro-Gln-Phe-Glu-Lys | n is 2 or 3 |
| 10 | WSHPQFEKGGGSGGGSGGGS | Twin-Strep-tag |
| | WSHPQFEK | |
| 11 | WSHPQFEKGGGSGGGSWSHP | Twin-Strep-tag |
| | QFEK | |
| 12 | WSHPQFEKGGGSGGGSGGSA | Twin-Strep-tag |
| | WSHPQFEK | |
| 13 | SAWSHPQFEKGGGSGGGSGG | Twin-Strep-tag |
| | GSWSHPQFEK | |
| 14 | SAWSHPQFEKGGGSGGGSGG | Twin-Strep-tag |
| | SAWSHPQFEK | |
| 15 | DPSKDSKAQVSAAEAGITGT | Streptavidin |
| | WYNQLGSTFIVTAGADGALT | Species: Streptomyces |
| | GTYESAVGNAESRYVLTGRY | avidinii |
| | DSAPATDGSGTALGWTVAWK | UniProt No. P22629 |
| | NNYRNAHSATTWSGQYVGGA | |
| | EARINTQWLLTSGTTEANAW | |
| | KSTLVGHDTFTKVKPSAASI | |
| | DAAKKAGVNNGNPLDAVQQ | |
| 16 | EAGITGTWYNQLGSTFIVTA | Mutein Streptavidin |
| | GADGALTGTYVTARGNAESR | Val44-Thr45-Ala46- |
| | YVLTGRYDSAPATDGSGTAL | Arg47 |
| | GWTVAWKNNYRNAHSATTWS | Species: Streptomyces |
| | GQYVGGAEARINTQWLLTSG | avidinii |
| | TTEANAWKSTLVGHDTFTKV | |
| | KPSAAS | |
| 17 | MEAGITGTWYNQLGSTFIVT | Mutein Streptavidin |
| | AGADGALTGTYVTARGNAES | Val44-Thr45-Ala46- |
| | RYVLTGRYDSAPATDGSGTA | Arg47 |
| | LGWTVAWKNNYRNAHSATTW | Species: Streptomyces |
| | SGQYVGGAEARINTQWLLTS | avidinii |
| | GTTEANAWKSTLVGHDTFTK | |
| | VKPSAAS | |
| 18 | DPSKDSKAQVSAAEAGITGT | Mutein Streptavidin |
| | WYNQLGSTFIVTAGADGALT | Ile44-Gly45-Ala-46- |
| | GTYIGARGNAESRYVLTGRY | Arg47 |
| | DSAPATDGSGTALGWTVAWK | Species: |
| | NNYRNAHSATTWSGQYVGGA | Streptomyces |
| | EARINTQWLLTSGTTEANAW | avidinii |
| | KSTLVGHDTFTKVKPSAASI | |
| | DAAKKAGVNNGNPLDAVQQ | |
| 19 | EAGITGTWYNQLGSTFIVTA | Mutein Streptavidin |
| | GADGALTGTYIGARGNAESR | Ile44-Gly45-Ala-46- |
| | YVLTGRYDSAPATDGSGTAL | Arg47 |
| | GWTVAWKNNYRNAHSATTWS | Species: Streptomyces |
| | GQYVGGAEARINTQWLLTSG | avidinii |
| | TTEANAWKSTLVGHDTFTKV | |
| | KPSAAS | |
| 20 | MEAGITGTWYNQLGSTFIVT | Mutein Streptavidin |
| | AGADGALTGTYIGARGNAES | Ile44-Gly45-Ala-46- |
| | RYVLTGRYDSAPATDGSGTA | Arg47 |
| | LGWTVAWKNNYRNAHSATTW | Species: Streptomyces |
| | SGQYVGGAEARINTQWLLTS | avidinii |
| | GTTEANAWKSTLVGHDTFTK | |
| | VKPSAAS | |
| 21 | EAGITGTWYNQLGSTFIVTA | Mutein Streptavidin |
| | GADGALTGTYVTARGNAESR | Val44-Thr45-Ala46- |
| | YVLTGRYDSAPATDGSGTAL | Arg47 and Glu 117, |
| | GWTVAWKNNYRNAHSATTWS | Gly 120, Tryl21 |
| | GQYVGGAEARINTQWLLTSG | (mutein ml-9) |
| | TTEENAGYSTLVGHDTFTKV | Species: |
| | KPSAAS | Streptomyces |
| | | avidinii |
| 22 | DPSKDSKAQVSAAEAGITGT | Mutein Streptavidin |
| | WYNQLGSTFIVTAGADGALT | Val44-Thr45-Ala46- |
| | GTYVTARGNAESRYVLTGRY | Arg47 and Glu 117, |
| | DSAPATDGSGTALGWTVAWK | Gly 120, Try121 |
| | NNYRNAHSATTWSGQYVGGA | (mutein m1-9) |
| | EARINTQWLLTSGTTEENAG | Species: |
| | YSTLVGHDTFTKVKPSAAS | Streptomyces |
| | | avidinii |
| 23 | MEAGITGTWYNQLGSTFIVT | Minimal streptavidin |
| | AGADGALTGTYESAVGNAES | Species: |
| | RYVLTGRYDSAPATDGSGTA | Streptomyces |
| | LGWTVAWKNNYRNAHSATTW | avidinii |
| | SGQYVGGAEARINTQWLLTS | |
| | GTTEANAWKSTLVGHDTFTK | |
| | VKPSAAS | |

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

1. An automated method for T cell scale down processing, comprising:
activating an input set of T cells by automatically contacting the input set of T cells obtained from one or more donors with one or more activation reagents to generate a set of activated T cells;
transducing the set of activated T cells by automatically contacting the set of activated T cells with a recombinant viral vector under conditions that promote viral infection of the set of activated T cells, wherein the recombinant viral vector comprises a nucleic acid that encodes a heterologous recombinant protein to generate a set of transduced T cells;
expanding the set of transduced T cells;
automatically recovering the set of transduced T cells from the expansion media; and
harvesting the set of transduced T cells by automatically cryopreserving the set of transduced T cells to generate a harvested set of transduced T cells.

2. The method of claim 1, further comprising inoculating the set of activated T cells by automatically transferring the set of activated T cells into inoculation media.

3. The method of claim 1, wherein activating comprises:
automatically washing the input set of T cells;
optionally, automatically obtaining testing samples of the washed input set of T cells for viable cell counting;
automatically contacting the washed input set of T cells with the one or more activation reagents; and
optionally, automatically obtaining testing samples of the set of activated T cells after contact with the one or more activation reagents for viable cell counting.

4. The method of claim 1, wherein transducing comprises:
optionally, automatically obtaining testing samples of the set of activated T cells for viable cell counting;
automatically preparing the set of activated T cells for spinoculation;
automatically spinoculating the set of activated T cells by contacting the set of activated T cells with the recombinant viral vector and applying a centrifugal force to the set of activated T cells; and
optionally, incubating or inoculating the set of activated T cells in an mammalian cell incubator post transduction.

5. The method of claim 1, wherein inoculating comprises:
optionally, automatically obtaining testing samples of the set of activated T cells after transducing for viable cell counting; and
inoculating the set of activated T cells by automatically transferring the set of activated T cells to expansion plates and placing the expansion plates containing the set of activated T cells in mammalian cell incubator.

6. The method of claim 1, wherein expanding further comprises:
obtaining testing samples of the set of transduced T cells for viable cell counting; and
automatically performing mock perfusion/cell culture media exchange.

7. The method of claim 1, further compressing debeading wherein debeading comprises:
automatically debeading the set of transduced T cells and/or the set of activated T cells by applying a magnetic field.

8. The method of claim 1, wherein harvesting comprises:
placing the set of transduced T cells in cryovials with cryopreservation media; and
placing the cryovials in a liquid nitrogen tank.

9. (canceled)

10. (canceled)

11. (canceled)

12. The method of claim 1, wherein the heterologous recombinant protein comprises a recombinant receptor.

13. The method of claim 12, wherein the recombinant receptor is capable of binding to a target antigen that is associated with, specific to, and/or expressed on a cell or tissue of a disease, disorder or condition.

14. The method of claim 13, wherein the disease, disorder or condition is an infectious disease or disorder, an autoimmune disease, an inflammatory disease, or a tumor or a cancer.

15. (canceled)

16. The method of claim 12, wherein the recombinant receptor is or comprises a functional non-TCR antigen receptor or a TCR or antigen-binding fragment thereof.

17. The method of claim 12, wherein the recombinant receptor is a chimeric antigen receptor (CAR).

18. The method of claim 1, wherein the recombinant viral vector comprises is a retroviral vector.

19. The method of claim 18, wherein the retroviral viral vector is a lentiviral vector or gammaretroviral vector.

20. (canceled)

21. (canceled)

22. An automated method for T cell scale down processing, comprising:
activating an input set of T cells by automatically contacting the input set of T cells obtained from one or more donors with one or more activation reagents to generate a set of activated T cells;
modifying the set of activated T cells by contacting the activated T cells with a recombinant polynucleotide under conditions that promote incorporation of the recombinant polynucleotide into the activated T cells, wherein the recombinant polynucleotide comprises a nucleic acid that encodes a heterologous recombinant protein to generate a set of modified T cells;
expanding the set of modified T cells;
recovering the set of modified T cells from the expansion media; and
harvesting the set modified T cells by automatically cryopreserving the set modified T cells to generate a harvested set of modified T cells.

23. The method of claim 22, wherein the modifying step includes transducing, electroporating, reagent-based transfecting, cell compression, or squeezing.

24. The method of claim 22, wherein one or more of the steps of the method are performed automatically and/or without intervention from an operator.

25. The method of claim 22, further comprising inoculating the set of activated T cells by automatically transferring the set of activated T cells into inoculation media.

26. The method of claim 22, wherein activating comprises:
automatically washing the input set of T cells;
optionally, automatically obtaining testing samples of the washed input set of T cells for viable cell counting;
automatically contacting the washed input set of T cells with the one or more activation reagents; and
optionally, automatically obtaining testing samples of the set of activated T cells after contact with the one or more activation reagents for viable cell counting.

27. The method of claim 23, wherein transducing comprises:
optionally, automatically obtaining testing samples of the set of activated T cells for viable cell counting;
automatically preparing the set of activated T cells for spinoculation;
automatically spinoculating the set of activated T cells by contacting the set of activated T cells with the recombinant viral vector and applying a centrifugal force to the set of activated T cells; and
optionally, incubating or inoculating the set of activated T cells in an mammalian cell incubator post transduction.

28. The method of claim 27, wherein inoculating comprises:
optionally, automatically obtaining testing samples of the set of activated T cells after transducing for viable cell counting; and
inoculating the set of activated T cells by automatically transferring the set of activated T cells to expansion plates and placing the expansion plates containing the set of activated T cells in mammalian cell incubator.

29. The method of claim 22, wherein expanding further comprises:
obtaining testing samples of the set of modified T cells for viable cell counting; and
automatically performing mock perfusion/cell culture media exchange.

30. The method of claim 22, further comprising debeading wherein debeading comprises:
automatically debeading the set of modified T cells and/or the set of activated T cells by applying a magnetic field.

31. The method of claim 22, wherein harvesting comprises:
placing the set of modified T cells in cryovials with cryopreservation media; and
placing the cryovials in a liquid nitrogen tank.

32. (canceled)

33. (canceled)

34. (canceled)

35. The method of claim 22, wherein the heterologous recombinant protein comprises a recombinant receptor.

36. The method of claim 35, wherein the recombinant receptor is capable of binding to a target antigen that is associated with, specific to, and/or expressed on a cell or tissue of a disease, disorder or condition.

37. The method of claim 36, wherein the disease, disorder or condition is an infectious disease or disorder, an autoimmune disease, an inflammatory disease, or a tumor or a cancer.

38. (canceled)

39. The method of claim 35, wherein the recombinant receptor is or comprises a functional non-TCR antigen receptor or a TCR or antigen-binding fragment thereof.

40. The method of claim 35, wherein the recombinant receptor is a chimeric antigen receptor (CAR).

41. (canceled)

42. (canceled)

43. A multiplex automated system for T cell transduction, comprising:
an automated liquid handling system, and
a control system in communication with the automated liquid handling system, comprising one or more processers programmed to control the automated liquid handling system to perform the unit processes of:
activating a set of T cells;
modifying the set of T cells;
debeading the set T cells;
inoculating the set of T cells;
expanding the set of T cells; and
harvesting the set of T cells.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)


SEQUENCE LISTING (23 sequences)

SEQ ID NO: 1 - protein, 159 aa, Streptomyces avidinii
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - protein, 126 aa, Streptomyces avidinii
EAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 3 - protein, 4 aa, Artificial Sequence
  Synthetic Streptavidin-binding peptide
HPQF

SEQ ID NO: 4 - protein, 8 aa, Artificial Sequence
  Synthetic streptavadin-binding peptide
  MISC_FEATURE (1)..(1): W, K or R
  MISC_FEATURE (2)..(2): Any amino acid
  MISC_FEATURE (7)..(7): G or E
  MISC_FEATURE (8)..(8): G, K or R
XXHPQFXX

SEQ ID NO: 5 - protein, 8 aa, Artificial Sequence
  Synthetic Streptavidin-binding peptide
  MISC_FEATURE (2)..(2): Any amino acid
  MISC_FEATURE (7)..(7): G or E
  MISC_FEATURE (8)..(8): G, K or R
WXHPQFXX

SEQ ID NO: 6 - protein, 8 aa, Artificial Sequence
  Synthetic Streptavidin-binding peptide, Strep-tag
WRHPQFGG

SEQ ID NO: 7 - protein, 8 aa, Artificial Sequence
  Synthetic  Strep-tag II
WSHPQFEK

SEQ ID NO: 8 - protein, 17 aa, Artificial Sequence
  Synthetic Sequential modules of streptavidin- binding peptide
  MISC_FEATURE (9)..(9): Any amino acid
  REPEAT (9)..(9): Repeated 8 or 12 times
WSHPQFEKXWSHPQFEK

SEQ ID NO: 9 - protein, 20 aa, Artificial Sequence
  Synthetic Sequential modules of streptavidin- binding peptide
  REPEAT (9)..(12): Repeated two or three times
WSHPQFEKGGGSWSHPQFEK

SEQ ID NO: 10 - protein, 28 aa, Artificial Sequence
  Synthetic Twin-Strep-peptide tag
WSHPQFEKGGGSGGGSGGGSWSHPQFEK

SEQ ID NO: 11 - protein, 24 aa, Artificial Sequence
  Synthetic Twin-Strep-peptide tag
WSHPQFEKGGGSGGGSWSHPQFEK

SEQ ID NO: 12 - protein, 28 aa, Artificial Sequence
  Synthetic Twin-Strep-peptide tag
WSHPQFEKGGGSGGGSGGSAWSHPQFEK

SEQ ID NO: 13 - protein, 30 aa, Artificial Sequence
  Synthetic Twin-Strep-peptide tag
SAWSHPQFEKGGGSGGGSGGGSWSHPQFEK

SEQ ID NO: 14 - protein, 30 aa, Artificial Sequence
  Synthetic Twin-Strep-peptide tag
SAWSHPQFEKGGGSGGGSGGSAWSHPQFEK

SEQ ID NO: 15 - protein, 159 aa, Streptomyces avidinii; identical to SEQ ID NO: 1

SEQ ID NO: 16 - protein, 126 aa, Streptomyces avidinii
  MUTAGEN (31)..(34)
EAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 17 - protein, 127 aa, Streptomyces avidinii
  MUTAGEN (32)..(35)
MEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 18 - protein, 159 aa, Streptomyces avidinii
  MUTAGEN (44)..(47)
DPSKDSKAQVSAAEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 19 - protein, 126 aa, Streptomyces avidinii
  MUTAGEN (34)..(37)
EAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 20 - protein, 127 aa, Streptomyces avidinii
  MUTAGEN (32)..(35)
MEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 21 - protein, 126 aa, Streptomyces avidinii
  MUTAGEN (31)..(34)
EAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 22 - protein, 139 aa, Streptomyces avidinii
  MUTAGEN (44)..(47)
DPSKDSKAQVSAAEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 23 - protein, 127 aa, Streptomyces avidinii
MEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS